SEQUENCE LISTING (419 sequences)

SEQ ID NO: 1 - DNA, 25 bp, Homo sapiens
gactacccatcatattcagtgtccc

SEQ ID NO: 2 - DNA, 25 bp, Homo sapiens
gtccctgttcctcactcagagagga

SEQ ID NO: 3 - DNA, 25 bp, Homo sapiens
gtcccttcatttcttgagactcaac

SEQ ID NO: 4 - DNA, 25 bp, Homo sapiens
ggtctcaagagttgtccctggaagg

SEQ ID NO: 5 - DNA, 25 bp, Homo sapiens
gggcagtctgcatctatttcaggtt

SEQ ID NO: 6 - DNA, 25 bp, Homo sapiens
tatttcaggttgtggctcttggttc

SEQ ID NO: 7 - DNA, 25 bp, Homo sapiens
ctcttggttctaggactcttacttc

SEQ ID NO: 8 - DNA, 25 bp, Homo sapiens
tctggctaagggctcagcttcttgg

SEQ ID NO: 9 - DNA, 25 bp, Homo sapiens
agcttcttgggacttcaaccatctt

SEQ ID NO: 10 - DNA, 25 bp, Homo sapiens
agtatggctttgtccctatgactca

SEQ ID NO: 11 - DNA, 25 bp, Homo sapiens
ctgggatatcatctctacagcctgc

SEQ ID NO: 12 - DNA, 25 bp, Homo sapiens
gaacacataatccccgtggtggtca

SEQ ID NO: 13 - DNA, 25 bp, Homo sapiens
cagctcctggttcgagtgatcgtgt

SEQ ID NO: 14 - DNA, 25 bp, Homo sapiens
tgatcgtgtgtcgctgcaacgtgga

SEQ ID NO: 15 - DNA, 25 bp, Homo sapiens
gcagtgcatgcgcaaggtgggccgc

SEQ ID NO: 16 - DNA, 25 bp, Homo sapiens
catgcccacgaagctgtcggcagtg

SEQ ID NO: 17 - DNA, 25 bp, Homo sapiens
ttttcacccactggaccatgtcaag

SEQ ID NO: 18 - DNA, 25 bp, Homo sapiens
gaagaaggacccggatcaaccagca

SEQ ID NO: 19 - DNA, 25 bp, Homo sapiens
gaaggcgactgtctgaatggcccag

SEQ ID NO: 20 - DNA, 25 bp, Homo sapiens
ggcccaggcagctctagctgggagc

SEQ ID NO: 21 - DNA, 25 bp, Homo sapiens
ctccatctgagtcccctgggagaga

SEQ ID NO: 22 - DNA, 25 bp, Homo sapiens
ggacaccaactttatggactgccca

SEQ ID NO: 23 - DNA, 25 bp, Homo sapiens
gattggactcacaattatcgcctgc

SEQ ID NO: 24 - DNA, 25 bp, Homo sapiens
ttatcgcctgctttgctgtgatagt

SEQ ID NO: 25 - DNA, 25 bp, Homo sapiens
agtggcgtgaagaagctgcattggc

SEQ ID NO: 26 - DNA, 25 bp, Homo sapiens
gaagctgcattggctgcacaggcta

SEQ ID NO: 27 - DNA, 25 bp, Homo sapiens
ctgaataccatccctgtcatcagca

SEQ ID NO: 28 - DNA, 25 bp, Homo sapiens
taccaaaatatctgccatggtttta

SEQ ID NO: 29 - DNA, 25 bp, Homo sapiens
tttatgtcgtgaacacctgtgtaac

SEQ ID NO: 30 - DNA, 25 bp, Homo sapiens
ccattacctcccaaacatagcagtt

SEQ ID NO: 31 - DNA, 25 bp, Homo sapiens
gcagtttttctgagtttcatcacct

SEQ ID NO: 32 - DNA, 25 bp, Homo sapiens
gagtttcatcacctttgattcattt

SEQ ID NO: 33 - DNA, 25 bp, Homo sapiens
tttgattcattttgcctgtttttga

SEQ ID NO: 34 - DNA, 25 bp, Homo sapiens
caacgtctaactggacttcccaaga

SEQ ID NO: 35 - DNA, 25 bp, Homo sapiens
gataaatggtaccagcgtcctctta

SEQ ID NO: 36 - DNA, 25 bp, Homo sapiens
aagatgccttaatccattccttgag

SEQ ID NO: 37 - DNA, 25 bp, Homo sapiens
ccttctgcttctgagttgcacatta

SEQ ID NO: 38 - DNA, 25 bp, Homo sapiens
gcttccagactctttgcgttggaga

SEQ ID NO: 39 - DNA, 25 bp, Homo sapiens
gttaggatcttcaagtcccatcata

SEQ ID NO: 40 - DNA, 25 bp, Homo sapiens
gggattgctttcatctaattctggc

SEQ ID NO: 41 - DNA, 25 bp, Homo sapiens
aggacctcaccaaaagatccagcct

SEQ ID NO: 42 - DNA, 25 bp, Homo sapiens
agcctcatacctacatcagacaaaa

SEQ ID NO: 43 - DNA, 25 bp, Homo sapiens
caaaatatcgccgttgttccttctg

SEQ ID NO: 44 - DNA, 25 bp, Homo sapiens
tttgctttggaaacacccactcact

SEQ ID NO: 45 - DNA, 25 bp, Homo sapiens
gatacttgctccattcatacacaac

SEQ ID NO: 46 - DNA, 25 bp, Homo sapiens
gcagtcactttgacaggttcctttt

SEQ ID NO: 47 - DNA, 25 bp, Homo sapiens
ggttccttttaagtggactctctga

SEQ ID NO: 48 - DNA, 25 bp, Homo sapiens
ggcttggatgtttttagttctgact

SEQ ID NO: 49 - DNA, 25 bp, Homo sapiens
ataatgactggtcttccttacctgg

SEQ ID NO: 50 - DNA, 25 bp, Homo sapiens
gttagttttagaattacaccacaag

SEQ ID NO: 51 - DNA, 25 bp, Homo sapiens
atattgttttgcattgtctgttggc

SEQ ID NO: 52 - DNA, 25 bp, Homo sapiens
gtgaactgtcatgatacgcttaagg

SEQ ID NO: 53 - DNA, 25 bp, Homo sapiens
atatatttttactgctttctgaatg

SEQ ID NO: 54 - DNA, 25 bp, Homo sapiens
gcttttttaactcatccgtttgcca

SEQ ID NO: 55 - DNA, 25 bp, Homo sapiens
actcatccgtttgccaatcattgca

SEQ ID NO: 56 - DNA, 25 bp, Homo sapiens
atctatgctaaaatgagccctttcc

SEQ ID NO: 57 - DNA, 25 bp, Homo sapiens
aagtcaccctatcaggtttcaaaca

SEQ ID NO: 58 - DNA, 25 bp, Homo sapiens
gaaatttcccttattcaggtttctg

SEQ ID NO: 59 - DNA, 25 bp, Homo sapiens
aatgaaagattaaccttccccactg

SEQ ID NO: 60 - DNA, 25 bp, Homo sapiens
actggtgatgacctaggcaggaatc

SEQ ID NO: 61 - DNA, 25 bp, Homo sapiens
ggcaagcaggtgtgaagagcagggc

SEQ ID NO: 62 - DNA, 25 bp, Homo sapiens
agcagggctcagcagcaagtcacat

SEQ ID NO: 63 - DNA, 25 bp, Homo sapiens
catttttctactatttgaccaaaag

SEQ ID NO: 64 - DNA, 25 bp, Homo sapiens
agaactctggagtggtctaagactg

SEQ ID NO: 65 - DNA, 25 bp, Homo sapiens
atcagaccaacagattttcccaacc

SEQ ID NO: 66 - DNA, 25 bp, Homo sapiens
gattttcccaacccaagactattgt

SEQ ID NO: 67 - DNA, 25 bp, Homo sapiens
ggaagcttcatctgaccaatgtggg

SEQ ID NO: 68 - DNA, 25 bp, Homo sapiens
cttgtgaaatgtgtccctaagcctc

SEQ ID NO: 69 - DNA, 25 bp, Homo sapiens
ccacccacccaggtgtctaagatag

SEQ ID NO: 70 - DNA, 25 bp, Homo sapiens
agataggacatgctcctttctttct

SEQ ID NO: 71 - DNA, 25 bp, Homo sapiens
tctctaatcccatcctgaggttgcc

SEQ ID NO: 72 - DNA, 25 bp, Homo sapiens
gcaaagccaatatgaccactactga

SEQ ID NO: 73 - DNA, 25 bp, Homo sapiens
aaatgcaagggtcttaccctcctct

SEQ ID NO: 74 - DNA, 25 bp, Homo sapiens
ttgccatcccccaaatgtgtggtat

SEQ ID NO: 75 - DNA, 25 bp, Homo sapiens
tggtatggtgaaactaatcccctga

SEQ ID NO: 76 - DNA, 25 bp, Homo sapiens
atcccctgaatgtgaattgctatcc

SEQ ID NO: 77 - DNA, 25 bp, Homo sapiens
ttgctatccttattgccctattaaa

SEQ ID NO: 78 - DNA, 25 bp, Homo sapiens
aagctttttgctgtccatgaatcct

SEQ ID NO: 79 - DNA, 25 bp, Homo sapiens
catgaatcctctgtggcaactgtaa

SEQ ID NO: 80 - DNA, 25 bp, Homo sapiens
gtcatgtctcttgtgccatggagct

SEQ ID NO: 81 - DNA, 25 bp, Homo sapiens
gctactgagtggtgctttatctgaa

SEQ ID NO: 82 - DNA, 25 bp, Homo sapiens
atgggactggctttccacaggaagt

SEQ ID NO: 83 - DNA, 25 bp, Homo sapiens
gtaaactgcttcagagcccacagtc

SEQ ID NO: 84 - DNA, 25 bp, Homo sapiens
ccctgctcagtgtccggaaagaagt

SEQ ID NO: 85 - DNA, 25 bp, Homo sapiens
ggaaagaagtcagtcatccctgttg

SEQ ID NO: 86 - DNA, 25 bp, Homo sapiens
ccctgttggcagtaaatcttcccac

SEQ ID NO: 87 - DNA, 25 bp, Homo sapiens
tcccacaggccgtccattagagatt

SEQ ID NO: 88 - DNA, 25 bp, Homo sapiens
gtgctatttcatatctgtactccag

SEQ ID NO: 89 - DNA, 25 bp, Homo sapiens
agcagatctcatgctctcagatagg

SEQ ID NO: 90 - DNA, 25 bp, Homo sapiens
gttcttttgtcacagtgctggctct

SEQ ID NO: 91 - DNA, 25 bp, Homo sapiens
cggggcttgcttggttaaccacaga

SEQ ID NO: 92 - DNA, 25 bp, Homo sapiens
ggaagcacggttactgtgttctcta

SEQ ID NO: 93 - DNA, 25 bp, Homo sapiens
taaagatgtaacctccacacctttc

SEQ ID NO: 94 - DNA, 25 bp, Homo sapiens
tccacacctttctccagattgggtg

SEQ ID NO: 95 - DNA, 25 bp, Homo sapiens
aaggtggtgggagtatctgtcgggg

SEQ ID NO: 96 - DNA, 25 bp, Homo sapiens
ggcccttggatgggtcaggtgggtg

SEQ ID NO: 97 - DNA, 25 bp, Homo sapiens
tgagctcaaagttgtcctactgcca

SEQ ID NO: 98 - DNA, 25 bp, Homo sapiens
tattttgcacttgttactgtaccat

SEQ ID NO: 99 - DNA, 25 bp, Homo sapiens
tctgtatgtgggatctgtgcttggg

SEQ ID NO: 100 - DNA, 25 bp, Homo sapiens
tgctgaggagtacgtgggcctgtct

SEQ ID NO: 101 - DNA, 25 bp, Homo sapiens
caaggacagggtggactgcggctac

SEQ ID NO: 102 - DNA, 25 bp, Homo sapiens
cccaaggagtgcaacaaccggggct

SEQ ID NO: 103 - DNA, 25 bp, Homo sapiens
tccaggatccctggagtgccttggt

SEQ ID NO: 104 - DNA, 25 bp, Homo sapiens
tggagtgccttggtgtttcaagccc

SEQ ID NO: 105 - DNA, 25 bp, Homo sapiens
gtttcaagcccctgactaggaagac

SEQ ID NO: 106 - DNA, 25 bp, Homo sapiens
cagaatgcaccttctgaggcacctc

SEQ ID NO: 107 - DNA, 25 bp, Homo sapiens
ctgtgattgctgccaggcactgttc

SEQ ID NO: 108 - DNA, 25 bp, Homo sapiens
attgctgccaggcactgttcatctc

SEQ ID NO: 109 - DNA, 25 bp, Homo sapiens
tcccggcaagctttctgctgaaagt

SEQ ID NO: 110 - DNA, 25 bp, Homo sapiens
aagttcatatctggagcctgatgtc

SEQ ID NO: 111 - DNA, 25 bp, Homo sapiens
gagaaattactcctcagattcttgc

SEQ ID NO: 112 - DNA, 25 bp, Homo sapiens
aatactcttaagtgcatgtaatcaa

SEQ ID NO: 113 - DNA, 25 bp, Homo sapiens
aggggtgtcactgttttcatcccct

SEQ ID NO: 114 - DNA, 25 bp, Homo sapiens
ggaccccaacttcagagggtgcagc

SEQ ID NO: 115 - DNA, 25 bp, Homo sapiens
gggtgcagcctctgaaagagactgt

SEQ ID NO: 116 - DNA, 25 bp, Homo sapiens
gagactgtgaatctatatataccat

SEQ ID NO: 117 - DNA, 25 bp, Homo sapiens
ttctgggacgaattcatcttctgag

SEQ ID NO: 118 - DNA, 25 bp, Homo sapiens
ttccatctgaattgcctcctagata

SEQ ID NO: 119 - DNA, 25 bp, Homo sapiens
aaatgctgcagctacattcttgcct

SEQ ID NO: 120 - DNA, 25 bp, Homo sapiens
ggactctagttcagttttatatgca

SEQ ID NO: 121 - DNA, 25 bp, Homo sapiens
atatgcaatggatcactattttatt

SEQ ID NO: 122 - DNA, 25 bp, Homo sapiens
ccatgaggcttgtccagacgcagtg

SEQ ID NO: 123 - DNA, 25 bp, Homo sapiens
aggcttgtccagacgcagtgaacca

SEQ ID NO: 124 - DNA, 25 bp, Homo sapiens
gtccagacgcagtgaaccagtcgca

SEQ ID NO: 125 - DNA, 25 bp, Homo sapiens
acgcagtgaaccagtcgcagctgat

SEQ ID NO: 126 - DNA, 25 bp, Homo sapiens
gaaccagtcgcagctgataacacac

SEQ ID NO: 127 - DNA, 25 bp, Homo sapiens
gtcgcagctgataacacacagacca

SEQ ID NO: 128 - DNA, 25 bp, Homo sapiens
ctgataacacacagaccattcccga

SEQ ID NO: 129 - DNA, 25 bp, Homo sapiens
tcccgatcccagaggtgcatttcag

SEQ ID NO: 130 - DNA, 25 bp, Homo sapiens
atcccagaggtgcatttcaggattc

SEQ ID NO: 131 - DNA, 25 bp, Homo sapiens
ttctatttcatcagagacggggtct

SEQ ID NO: 132 - DNA, 25 bp, Homo sapiens
ggcctcccagagggttggaattgca

SEQ ID NO: 133 - DNA, 25 bp, Homo sapiens
gaattcgacacaatggggttgcgtc

SEQ ID NO: 134 - DNA, 25 bp, Homo sapiens
ggggttgcgtcacctatagttttca

SEQ ID NO: 135 - DNA, 25 bp, Homo sapiens
gcaactcaaggtgctcagcttgata

SEQ ID NO: 136 - DNA, 25 bp, Homo sapiens
ggcaggggagaaactgactcctttt

SEQ ID NO: 137 - DNA, 25 bp, Homo sapiens
tctcttttttgcctgcacacttaat

SEQ ID NO: 138 - DNA, 25 bp, Homo sapiens
cacacttaatgtggactgcctcctt

SEQ ID NO: 139 - DNA, 25 bp, Homo sapiens
tggactgcctccttggacttaaaaa

SEQ ID NO: 140 - DNA, 25 bp, Homo sapiens
atttcttagctttggacttgacaac

SEQ ID NO: 141 - DNA, 25 bp, Homo sapiens
gacaactgacgaatgctctgggggt

SEQ ID NO: 142 - DNA, 25 bp, Homo sapiens
gtgtttatgttgtcacttcgcttgc

SEQ ID NO: 143 - DNA, 25 bp, Homo sapiens
cacctcacccgatcttggttataag

SEQ ID NO: 144 - DNA, 25 bp, Homo sapiens
ggatctgctcgtcatcatttttcct

SEQ ID NO: 145 - DNA, 25 bp, Homo sapiens
tcacgaatattatgcccagtttctg

SEQ ID NO: 146 - DNA, 25 bp, Homo sapiens
atattatgcccagtttctgcctctg

SEQ ID NO: 147 - DNA, 25 bp, Homo sapiens
attatgcccagtttctgcctctgag

SEQ ID NO: 148 - DNA, 25 bp, Homo sapiens
ttatgcccagtttctgcctctgagg

SEQ ID NO: 149 - DNA, 25 bp, Homo sapiens
tttctgcctctgagggaaagcccag

SEQ ID NO: 150 - DNA, 25 bp, Homo sapiens
cctctgagggaaagcccagaaaagg

SEQ ID NO: 151 - DNA, 25 bp, Homo sapiens
aggggcccagtcctggcctggcttc

SEQ ID NO: 152 - DNA, 25 bp, Homo sapiens
tggcctggcttctcctttggaagtg

SEQ ID NO: 153 - DNA, 25 bp, Homo sapiens
tggcttctcctttggaagtgaggca

SEQ ID NO: 154 - DNA, 25 bp, Homo sapiens
tcctttggaagtgaggcattgcacg

SEQ ID NO: 155 - DNA, 25 bp, Homo sapiens
cctttggaagtgaggcattgcacgg

SEQ ID NO: 156 - DNA, 25 bp, Homo sapiens
cattgcacggggagacgtacgtatc

SEQ ID NO: 157 - DNA, 25 bp, Homo sapiens
ttgcacggggagacgtacgtatcag

SEQ ID NO: 158 - DNA, 25 bp, Homo sapiens
gacgtacgtatcagcggccccttga

SEQ ID NO: 159 - DNA, 25 bp, Homo sapiens
atcagcggccccttgactctgggga

SEQ ID NO: 160 - DNA, 25 bp, Homo sapiens
ggcatccactctgcggaggaatgac

SEQ ID NO: 161 - DNA, 25 bp, Homo sapiens
gaggaatgactccatctacgaggcc

SEQ ID NO: 162 - DNA, 25 bp, Homo sapiens
gaggagtataactacggcttcgtgg

SEQ ID NO: 163 - DNA, 25 bp, Homo sapiens
gtataactacggcttcgtggtggag

SEQ ID NO: 164 - DNA, 25 bp, Homo sapiens
aactacggcttcgtggtggagaaga

SEQ ID NO: 165 - DNA, 25 bp, Homo sapiens
cttcgccctgcaaggcgtttgctat

SEQ ID NO: 166 - DNA, 25 bp, Homo sapiens
cgccctgcaaggcgtttgctatctt

SEQ ID NO: 167 - DNA, 25 bp, Homo sapiens
gcaaggcgtttgctatcttcagcca

SEQ ID NO: 168 - DNA, 25 bp, Homo sapiens
ggagctggacagaagccagtgcctt

SEQ ID NO: 169 - DNA, 25 bp, Homo sapiens
tggacagaagccagtgcctttaagt

SEQ ID NO: 170 - DNA, 25 bp, Homo sapiens
ctcctggcaataaacactacccggt

SEQ ID NO: 171 - DNA, 25 bp, Homo sapiens
cggttcttcagttttctgagaagag

SEQ ID NO: 172 - DNA, 25 bp, Homo sapiens
tcgcctctgcaggaaagagcagagc

SEQ ID NO: 173 - DNA, 25 bp, Homo sapiens
cctctgcaggaaagagcagagcccg

SEQ ID NO: 174 - DNA, 25 bp, Homo sapiens
gcccagcacgcatggtattgccagc

SEQ ID NO: 175 - DNA, 25 bp, Homo sapiens
tgtagtctctgctccctgcatttca

SEQ ID NO: 176 - DNA, 25 bp, Homo sapiens
gactggcaacagaacagccgtggct

SEQ ID NO: 177 - DNA, 25 bp, Homo sapiens
aacagccgtggctgctttatctctc

SEQ ID NO: 178 - DNA, 25 bp, Homo sapiens
gggagcctggccacctcgcagctgt

SEQ ID NO: 179 - DNA, 25 bp, Homo sapiens
ggagcctggccacctcgcagctgtt

SEQ ID NO: 180 - DNA, 25 bp, Homo sapiens
ccctcaacctttgaactggaactgc

SEQ ID NO: 181 - DNA, 25 bp, Homo sapiens
aacctttgaactggaactgctggct

SEQ ID NO: 182 - DNA, 25 bp, Homo sapiens
agggttttcgacaactgcagctgaa

SEQ ID NO: 183 - DNA, 25 bp, Homo sapiens
ttttcgacaactgcagctgaatctc

SEQ ID NO: 184 - DNA, 25 bp, Homo sapiens
gacaactgcagctgaatctcatgga

SEQ ID NO: 185 - DNA, 25 bp, Homo sapiens
tgaatctcatggaaaagctggattc

SEQ ID NO: 186 - DNA, 25 bp, Homo sapiens
ggaaaagctggattcctctgcctta

SEQ ID NO: 187 - DNA, 25 bp, Homo sapiens
aggagggaagcctctgtgctgctca

SEQ ID NO: 188 - DNA, 25 bp, Homo sapiens
gggaagcctctgtgctgctcacctg

SEQ ID NO: 189 - DNA, 25 bp, Homo sapiens
aaagcccagctcagcgggtacgcag

SEQ ID NO: 190 - DNA, 25 bp, Homo sapiens
cgcagtggaggcgacaaggaaccaa

SEQ ID NO: 191 - DNA, 25 bp, Homo sapiens
ggaggcgacaaggaaccaatcacaa

SEQ ID NO: 192 - DNA, 25 bp, Homo sapiens
gacaaggaaccaatcacaactacag

SEQ ID NO: 193 - DNA, 25 bp, Homo sapiens
caatcacaactacagaggtttctct

SEQ ID NO: 194 - DNA, 25 bp, Homo sapiens
tacagaggtttctctgatcctcctc

SEQ ID NO: 195 - DNA, 25 bp, Homo sapiens
gaggtttctctgatcctcctccact

SEQ ID NO: 196 - DNA, 25 bp, Homo sapiens
actcccgctgcttcaacttgactaa

SEQ ID NO: 197 - DNA, 25 bp, Homo sapiens
gctgcttcaacttgactaagcttaa

SEQ ID NO: 198 - DNA, 25 bp, Homo sapiens
tgcgtcttcccaagttagcaggtgt

SEQ ID NO: 199 - DNA, 25 bp, Homo sapiens
tcctggcagaagccatggcccatgt

SEQ ID NO: 200 - DNA, 25 bp, Homo sapiens
agaaggcccagctcacatgtgagtc

SEQ ID NO: 201 - DNA, 25 bp, Homo sapiens
aggcccagctcacatgtgagtcctg

SEQ ID NO: 202 - DNA, 25 bp, Homo sapiens
cagaagccatggcccatgtctgcac

SEQ ID NO: 203 - DNA, 25 bp, Homo sapiens
aaggcccagctcacatgtgagtcct

SEQ ID NO: 204 - DNA, 25 bp, Homo sapiens
gcagaagccatggcccatgtctgca

SEQ ID NO: 205 - DNA, 25 bp, Homo sapiens
cccacgcccgaacagcaggggcaga

SEQ ID NO: 206 - DNA, 25 bp, Homo sapiens
tccttcgaagtgtgtccaagtccgc

SEQ ID NO: 207 - DNA, 25 bp, Homo sapiens
tgtccaagtccgcatttgagccttg

SEQ ID NO: 208 - DNA, 25 bp, Homo sapiens
ggctcactgtcctgagttgcagtaa

SEQ ID NO: 209 - DNA, 25 bp, Homo sapiens
aggcccagccggaacgagatttacg

SEQ ID NO: 210 - DNA, 25 bp, Homo sapiens
agtgcgcatgcgtagatggagcaaa

SEQ ID NO: 211 - DNA, 25 bp, Homo sapiens
tagatggagcaaaaccgcctctcca

SEQ ID NO: 212 - DNA, 25 bp, Homo sapiens
atcgacccttgggtccagcaaggaa

SEQ ID NO: 213 - DNA, 25 bp, Homo sapiens
cttgggtccagcaaggaagccctaa

SEQ ID NO: 214 - DNA, 25 bp, Homo sapiens
ggaagccctaagtgacgtcaacctt

SEQ ID NO: 215 - DNA, 25 bp, Homo sapiens
gacgtcaacctttgttcccatggta

SEQ ID NO: 216 - DNA, 25 bp, Homo sapiens
ttcccatggtaacgcagaaccttgg

SEQ ID NO: 217 - DNA, 25 bp, Homo sapiens
cggaaaaccggctcagttgacgctg

SEQ ID NO: 218 - DNA, 25 bp, Homo sapiens
cggctcagttgacgctgcaggtaaa

SEQ ID NO: 219 - DNA, 25 bp, Homo sapiens
gttgacgctgcaggtaaaaagactt

SEQ ID NO: 220 - DNA, 25 bp, Homo sapiens
gtcacgggatggatagccggcctgc

SEQ ID NO: 221 - DNA, 25 bp, Homo sapiens
atattgtgaacgagccacctcctaa

SEQ ID NO: 222 - DNA, 25 bp, Homo sapiens
catcaagaacccagcggagcgggcg

SEQ ID NO: 223 - DNA, 25 bp, Homo sapiens
gacctgaagatgctcacaaaccaca

SEQ ID NO: 224 - DNA, 25 bp, Homo sapiens
acaaaccacaccttcatcaagcggt

SEQ ID NO: 225 - DNA, 25 bp, Homo sapiens
ccttcatcaagcggtccgaggtgga

SEQ ID NO: 226 - DNA, 25 bp, Homo sapiens
gaagtggattttgccggctggttgt

SEQ ID NO: 227 - DNA, 25 bp, Homo sapiens
tgtgtaaaaccctgcggctgaacca

SEQ ID NO: 228 - DNA, 25 bp, Homo sapiens
acagcgcatgcaggaacgggggtct

SEQ ID NO: 229 - DNA, 25 bp, Homo sapiens
tgctgtgtgtggtctcagaggctct

SEQ ID NO: 230 - DNA, 25 bp, Homo sapiens
ctctgcttccttaggttacaaaaca

SEQ ID NO: 231 - DNA, 25 bp, Homo sapiens
gtgacaacgtagagcgcgtgctgaa

SEQ ID NO: 232 - DNA, 25 bp, Homo sapiens
tgaaggtgctcacgggcacgggtaa

SEQ ID NO: 233 - DNA, 25 bp, Homo sapiens
tattcagcctaactatcctgcggca

SEQ ID NO: 234 - DNA, 25 bp, Homo sapiens
ctttgccctgaacttgtccgggtag

SEQ ID NO: 235 - DNA, 25 bp, Homo sapiens
ggagggccggaggcatgtggcctcc

SEQ ID NO: 236 - DNA, 25 bp, Homo sapiens
gacctcctgacctgggtggttgagg

SEQ ID NO: 237 - DNA, 25 bp, Homo sapiens
ggttgaggctcaagacagctcaccc

SEQ ID NO: 238 - DNA, 25 bp, Homo sapiens
ggtccagaagctccatgctggtcac

SEQ ID NO: 239 - DNA, 25 bp, Homo sapiens
atgctggtcactagggtgctgtgct

SEQ ID NO: 240 - DNA, 25 bp, Homo sapiens
ggcccaagcaggtgggagtggacac

SEQ ID NO: 241 - DNA, 25 bp, Homo sapiens
cctgctcaggcctcttgagaaatgg

SEQ ID NO: 242 - DNA, 25 bp, Homo sapiens
gccgcaccctgagcatggacaaggg

SEQ ID NO: 243 - DNA, 25 bp, Homo sapiens
ggacaagggcttctgaggccagcca

SEQ ID NO: 244 - DNA, 25 bp, Homo sapiens
gcgccagggctcagatggggcctgg

SEQ ID NO: 245 - DNA, 25 bp, Homo sapiens
tgggctgttgtgtctggagcctgca

SEQ ID NO: 246 - DNA, 25 bp, Homo sapiens
gagcctgcaggcaggtgggggctgc

SEQ ID NO: 247 - DNA, 25 bp, Homo sapiens
cctccggctggtgtcttgagaccag

SEQ ID NO: 248 - DNA, 25 bp, Homo sapiens
ggacagagtgccctggggcatccac

SEQ ID NO: 249 - DNA, 25 bp, Homo sapiens
gctgggctcactacatgggccagcc

SEQ ID NO: 250 - DNA, 25 bp, Homo sapiens
tgctggcgcctaggttgtgttgaga

SEQ ID NO: 251 - DNA, 25 bp, Homo sapiens
cacctgtcaccttttctagaaataa

SEQ ID NO: 252 - DNA, 25 bp, Homo sapiens
accctgactgtggggtgcatcggtc

SEQ ID NO: 253 - DNA, 25 bp, Homo sapiens
gaggcttctccagagctgggagctg

SEQ ID NO: 254 - DNA, 25 bp, Homo sapiens
tgtcacccagccatggtgtggtcca

SEQ ID NO: 255 - DNA, 25 bp, Homo sapiens
tccaggcagggacatctcggtaccc

SEQ ID NO: 256 - DNA, 25 bp, Homo sapiens
tcgtccacaaacactccgtggctga

SEQ ID NO: 257 - DNA, 25 bp, Homo sapiens
ggctgagaggcagcggatccaggca

SEQ ID NO: 258 - DNA, 25 bp, Homo sapiens
atccaggcagcgatgctgagccacc

SEQ ID NO: 259 - DNA, 25 bp, Homo sapiens
accaccccggaggacacgtggatga

SEQ ID NO: 260 - DNA, 25 bp, Homo sapiens
tgatggggtcagagatcactgagct

SEQ ID NO: 261 - DNA, 25 bp, Homo sapiens
gcctccgtggctccaaggtgagggt

SEQ ID NO: 262 - DNA, 25 bp, Homo sapiens
ggtgagggtcatcttcacgagcaaa

SEQ ID NO: 263 - DNA, 25 bp, Homo sapiens
agtgacaacgaacgtctgaggcttc

SEQ ID NO: 264 - DNA, 25 bp, Homo sapiens
ctccgaaggcccagggaacatacct

SEQ ID NO: 265 - DNA, 25 bp, Homo sapiens
ccagcgccaggtgctcttgcctttt

SEQ ID NO: 266 - DNA, 25 bp, Homo sapiens
agcgccaggtgctcttgccttttcc

SEQ ID NO: 267 - DNA, 25 bp, Homo sapiens
gctcttgccttttcccactgagaga

SEQ ID NO: 268 - DNA, 25 bp, Homo sapiens
gccttttcccactgagagaaggctg

SEQ ID NO: 269 - DNA, 25 bp, Homo sapiens
ttcccactgagagaaggctgctctt

SEQ ID NO: 270 - DNA, 25 bp, Homo sapiens
tcccactgagagaaggctgctcttt

SEQ ID NO: 271 - DNA, 25 bp, Homo sapiens
ctgagagaaggctgctcttttgtac

SEQ ID NO: 272 - DNA, 25 bp, Homo sapiens
tgagagaaggctgctcttttgtact

SEQ ID NO: 273 - DNA, 25 bp, Homo sapiens
gagagaaggctgctcttttgtactg

SEQ ID NO: 274 - DNA, 25 bp, Homo sapiens
agagaaggctgctcttttgtactgc

SEQ ID NO: 275 - DNA, 25 bp, Homo sapiens
gagaaggctgctcttttgtactgcc

SEQ ID NO: 276 - DNA, 25 bp, Homo sapiens
agaaggctgctcttttgtactgccc

SEQ ID NO: 277 - DNA, 25 bp, Homo sapiens
aggggtggccagagccaaagccagc

SEQ ID NO: 278 - DNA, 25 bp, Homo sapiens
tggccagagccaaagccagctactg

SEQ ID NO: 279 - DNA, 25 bp, Homo sapiens
ccctcctgcattcctcatttaagga

SEQ ID NO: 280 - DNA, 25 bp, Homo sapiens
tattgagcaccctttgtgtgcagac

SEQ ID NO: 281 - DNA, 25 bp, Homo sapiens
accctttgtgtgcagacatggctcc

SEQ ID NO: 282 - DNA, 25 bp, Homo sapiens
gacatggctccaggtgcttagcaat

SEQ ID NO: 283 - DNA, 25 bp, Homo sapiens
gcttagcaatcaatggtgagcgtgg

SEQ ID NO: 284 - DNA, 25 bp, Homo sapiens
gtgagcgtggtattccaggctaaag

SEQ ID NO: 285 - DNA, 25 bp, Homo sapiens
attacaggctggttgtggcagctca

SEQ ID NO: 286 - DNA, 25 bp, Homo sapiens
tggttgtggcagctcatgactgtaa

SEQ ID NO: 287 - DNA, 25 bp, Homo sapiens
gtgctaacagctctagctactcagg

SEQ ID NO: 288 - DNA, 25 bp, Homo sapiens
gcagcatcaaccatctctgagataa

SEQ ID NO: 289 - DNA, 25 bp, Homo sapiens
aacaacttgcggctatccagatggc

SEQ ID NO: 290 - DNA, 25 bp, Homo sapiens
atggccgggcaatggcagggcaggt

SEQ ID NO: 291 - DNA, 25 bp, Homo sapiens
gcagggcaggtgccgtggccctgca

SEQ ID NO: 292 - DNA, 25 bp, Homo sapiens
gagaatgatctggtgcgaagcccca

SEQ ID NO: 293 - DNA, 25 bp, Homo sapiens
tgcaccaggctgttgaggacctgcc

SEQ ID NO: 294 - DNA, 25 bp, Homo sapiens
tgaggacctgccagagggcgtggac

SEQ ID NO: 295 - DNA, 25 bp, Homo sapiens
ccgcagggagttctatctctcagac

SEQ ID NO: 296 - DNA, 25 bp, Homo sapiens
cagactctgacttccaagatatctt

SEQ ID NO: 297 - DNA, 25 bp, Homo sapiens
tctacagcatggccacgtggaggca

SEQ ID NO: 298 - DNA, 25 bp, Homo sapiens
ggtcatcctctgcgtgtcagtaaaa

SEQ ID NO: 299 - DNA, 25 bp, Homo sapiens
cagtcagagtggacaaacctctgtg

SEQ ID NO: 300 - DNA, 25 bp, Homo sapiens
gacaaacctctgtgggaggctctac

SEQ ID NO: 301 - DNA, 25 bp, Homo sapiens
tctgtgggaggctctactgcactga

SEQ ID NO: 302 - DNA, 25 bp, Homo sapiens
ggaggctctactgcactgagatgca

SEQ ID NO: 303 - DNA, 25 bp, Homo sapiens
tgcactgagatgcagctcttccgag

SEQ ID NO: 304 - DNA, 25 bp, Homo sapiens
ggggctcctaagccagtgtacaact

SEQ ID NO: 305 - DNA, 25 bp, Homo sapiens
agtgtacaactgggtgcgtcttgga

SEQ ID NO: 306 - DNA, 25 bp, Homo sapiens
gtacaactgggtgcgtcttggaact

SEQ ID NO: 307 - DNA, 25 bp, Homo sapiens
caactgggtgcgtcttggaactttt

SEQ ID NO: 308 - DNA, 25 bp, Homo sapiens
gccaccaaccagatgggcagtgcat

SEQ ID NO: 309 - DNA, 25 bp, Homo sapiens
aaccagatgggcagtgcatcctgtg

SEQ ID NO: 310 - DNA, 25 bp, Homo sapiens
catgctgagctcagacttcaggccc

SEQ ID NO: 311 - DNA, 25 bp, Homo sapiens
ccacttcaacaagcacctgctgggc

SEQ ID NO: 312 - DNA, 25 bp, Homo sapiens
cagcagggctcggagcccagctacc

SEQ ID NO: 313 - DNA, 25 bp, Homo sapiens
ccgtcctgtgcagcaccatggagaa

SEQ ID NO: 314 - DNA, 25 bp, Homo sapiens
atggagaagagcgtgctcggcggcc

SEQ ID NO: 315 - DNA, 25 bp, Homo sapiens
tgcgcgtccgtgtgacggagctgga

SEQ ID NO: 316 - DNA, 25 bp, Homo sapiens
gaggacgaggtgcgcaacctgcgca

SEQ ID NO: 317 - DNA, 25 bp, Homo sapiens
acctgcgcaagatcaatcgggacct

SEQ ID NO: 318 - DNA, 25 bp, Homo sapiens
atcaatcgggacctgttcgacttct

SEQ ID NO: 319 - DNA, 25 bp, Homo sapiens
ggcacgcgtcacagtggtgctagtc

SEQ ID NO: 320 - DNA, 25 bp, Homo sapiens
agtggtgctagtctgtttttaacaa

SEQ ID NO: 321 - DNA, 25 bp, Homo sapiens
agaaatgcctcacagctatcgtgaa

SEQ ID NO: 322 - DNA, 25 bp, Homo sapiens
gctatcgtgaagtgcgccacaagca

SEQ ID NO: 323 - DNA, 25 bp, Homo sapiens
gcgccacaagcaaaccagctttctt

SEQ ID NO: 324 - DNA, 25 bp, Homo sapiens
aaaccagctttctttgcagagaagc

SEQ ID NO: 325 - DNA, 25 bp, Homo sapiens
gcagagaagcttcatcaagccatga

SEQ ID NO: 326 - DNA, 25 bp, Homo sapiens
ggtgttggaactcgccataaggcat

SEQ ID NO: 327 - DNA, 25 bp, Homo sapiens
tatggtttcccgttctgaaattgac

SEQ ID NO: 328 - DNA, 25 bp, Homo sapiens
agaagatgtatggtatctccctttg

SEQ ID NO: 329 - DNA, 25 bp, Homo sapiens
cctttgccaagccatcctggatgaa

SEQ ID NO: 330 - DNA, 25 bp, Homo sapiens
gaaactaaacattcccttgatggtc

SEQ ID NO: 331 - DNA, 25 bp, Homo sapiens
gatggtctcaagctatgatcagaag

SEQ ID NO: 332 - DNA, 25 bp, Homo sapiens
ctgtgccatctttcactgggaggca

SEQ ID NO: 333 - DNA, 25 bp, Homo sapiens
gtttttcatcctgccatatgacacc

SEQ ID NO: 334 - DNA, 25 bp, Homo sapiens
cacagtgaggagtagccaggttgct

SEQ ID NO: 335 - DNA, 25 bp, Homo sapiens
gttgctaggacagtaaccctgccac

SEQ ID NO: 336 - DNA, 25 bp, Homo sapiens
tgccacacactgcctgaaatcggaa

SEQ ID NO: 337 - DNA, 25 bp, Homo sapiens
gcctgaaatcggaactcccttggcc

SEQ ID NO: 338 - DNA, 25 bp, Homo sapiens
cctccctcttaactaagtgacccat

SEQ ID NO: 339 - DNA, 25 bp, Homo sapiens
gggaagggtattggacacggcagcg

SEQ ID NO: 340 - DNA, 25 bp, Homo sapiens
gcagcgtcctccttattgaaaacac

SEQ ID NO: 341 - DNA, 25 bp, Homo sapiens
atacatttttcatctcatggctaca

SEQ ID NO: 342 - DNA, 25 bp, Homo sapiens
tactgatgatccttccacttatatt

SEQ ID NO: 343 - DNA, 25 bp, Homo sapiens
gaatgtatttgctgacaaccattaa

SEQ ID NO: 344 - DNA, 25 bp, Homo sapiens
gaagttttactccaggttattgcaa

SEQ ID NO: 345 - DNA, 25 bp, Homo sapiens
tcatgtcccagaacttagcctttac

SEQ ID NO: 346 - DNA, 25 bp, Homo sapiens
ttagcctttacctgtgaagtgttac

SEQ ID NO: 347 - DNA, 25 bp, Homo sapiens
gaagtgttactacagccttaatatt

SEQ ID NO: 348 - DNA, 25 bp, Homo sapiens
tacagccttaatattttcctagtag

SEQ ID NO: 349 - DNA, 25 bp, Homo sapiens
gtgtttttagctgtgacacaactgt

SEQ ID NO: 350 - DNA, 25 bp, Homo sapiens
taactcaaggataccttcttattta

SEQ ID NO: 351 - DNA, 25 bp, Homo sapiens
aacatttatatgtagcctttactgt

SEQ ID NO: 352 - DNA, 25 bp, Homo sapiens
tagcctttactgtttgatataccaa

SEQ ID NO: 353 - DNA, 25 bp, Homo sapiens
ctcattacttatactgggacaccat

SEQ ID NO: 354 - DNA, 25 bp, Homo sapiens
acagctttccaaggtgacaaactcc

SEQ ID NO: 355 - DNA, 25 bp, Homo sapiens
aaactcctctatgcagtgtatcgaa

SEQ ID NO: 356 - DNA, 25 bp, Homo sapiens
atagccatatggggctggtcctatg

SEQ ID NO: 357 - DNA, 25 bp, Homo sapiens
tggaggatacgtttcatcactggcc

SEQ ID NO: 358 - DNA, 25 bp, Homo sapiens
ccttgcatctggaactggtcttttc

SEQ ID NO: 359 - DNA, 25 bp, Homo sapiens
tggtatagcagtggctccagtctcc

SEQ ID NO: 360 - DNA, 25 bp, Homo sapiens
gaatattacgcgtctgtctacacag

SEQ ID NO: 361 - DNA, 25 bp, Homo sapiens
gagagattcatgggtctcccaacaa

SEQ ID NO: 362 - DNA, 25 bp, Homo sapiens
aatgtagactatcttctcatccacg

SEQ ID NO: 363 - DNA, 25 bp, Homo sapiens
tgaccagaaccacggcttatccggc

SEQ ID NO: 364 - DNA, 25 bp, Homo sapiens
acatgacccacttcctaaagcagtg

SEQ ID NO: 365 - DNA, 25 bp, Homo sapiens
gggagtttcctgagggttttctcat

SEQ ID NO: 366 - DNA, 25 bp, Homo sapiens
aaatgagggctgcacattgcctgtt

SEQ ID NO: 367 - DNA, 25 bp, Homo sapiens
attgcctgttctgcttcgaagtatt

SEQ ID NO: 368 - DNA, 25 bp, Homo sapiens
gaaagcatatgcagccaaccaagat

SEQ ID NO: 369 - DNA, 25 bp, Homo sapiens
aaagtcacccaaacacttctgcttt

SEQ ID NO: 370 - DNA, 25 bp, Homo sapiens
tgctttcacttaagtgtctggcccg

SEQ ID NO: 371 - DNA, 25 bp, Homo sapiens
gtctggcccgcaatactgtaggaac

SEQ ID NO: 372 - DNA, 25 bp, Homo sapiens
aatatccacagtactcactttttcc

SEQ ID NO: 373 - DNA, 25 bp, Homo sapiens
gtactcactttttccaaatgatcct

SEQ ID NO: 374 - DNA, 25 bp, Homo sapiens
gaaatatctttctcttacctgttat

SEQ ID NO: 375 - DNA, 25 bp, Homo sapiens
tatgtgccaaagctttactactgtg

SEQ ID NO: 376 - DNA, 25 bp, Homo sapiens
aagtttggtatgtttctctcaagaa

SEQ ID NO: 377 - DNA, 25 bp, Homo sapiens
aaagcatggtgagtccagacagcag

SEQ ID NO: 378 - DNA, 25 bp, Homo sapiens
tggaaactccctgttagtacgtcct

SEQ ID NO: 379 - DNA, 25 bp, Homo sapiens
ggtggttattgatggcgttgccttt

SEQ ID NO: 380 - DNA, 25 bp, Homo sapiens
gttgcctttgtttcatctttgagtt

SEQ ID NO: 381 - DNA, 25 bp, Homo sapiens
tcatctttgagtttgccctttgtgg

SEQ ID NO: 382 - DNA, 25 bp, Homo sapiens
gtttgccctttgtgggatctagtgg

SEQ ID NO: 383 - DNA, 25 bp, Homo sapiens
gagcactgacagaactcttaacagc

SEQ ID NO: 384 - DNA, 25 bp, Homo sapiens
aacagcgtgctgtatttttgacatt

SEQ ID NO: 385 - DNA, 25 bp, Homo sapiens
tgacttgatttgtacataactctgt

SEQ ID NO: 386 - DNA, 25 bp, Homo sapiens
gtttttgtaccttagaatttctgca

SEQ ID NO: 387 - DNA, 25 bp, Homo sapiens
ctactactatacggctgcgagaaga

SEQ ID NO: 388 - DNA, 25 bp, Homo sapiens
atacggctgcgagaagacgacagaa

SEQ ID NO: 389 - DNA, 25 bp, Homo sapiens
gacgacagaagggctacgacgtggt

SEQ ID NO: 390 - DNA, 25 bp, Homo sapiens
agaagggctacgacgtggtggcggt

SEQ ID NO: 391 - DNA, 25 bp, Homo sapiens
ggtgtaccccaatgccaaggtggaa

SEQ ID NO: 392 - DNA, 25 bp, Homo sapiens
gtaccccaatgccaaggtggaactc

SEQ ID NO: 393 - DNA, 25 bp, Homo sapiens
atgccaaggtggaactcgtctgagc

SEQ ID NO: 394 - DNA, 25 bp, Homo sapiens
aggtggaactcgtctgagctgggac

SEQ ID NO: 395 - DNA, 25 bp, Homo sapiens
ccctgctcggccggtgtaaatatgt

SEQ ID NO: 396 - DNA, 25 bp, Homo sapiens
tgctcggccggtgtaaatatgtttc

SEQ ID NO: 397 - DNA, 25 bp, Homo sapiens
gtgtaaatatgtttcaccctgtccc

SEQ ID NO: 398 - DNA, 25 bp, Homo sapiens
aagacacttcttccaaaccttgaat

SEQ ID NO: 399 - DNA, 25 bp, Homo sapiens
gatgtgtgtttacttcatgtttaca

SEQ ID NO: 400 - DNA, 25 bp, Homo sapiens
atcagccaaaaccataacttacaat

SEQ ID NO: 401 - DNA, 25 bp, Homo sapiens
ttggatatgctttaccattcttagg

SEQ ID NO: 402 - DNA, 25 bp, Homo sapiens
accattcttaggtttctgtggaaca

SEQ ID NO: 403 - DNA, 25 bp, Homo sapiens
tttttccaattgctattgcccaaga

SEQ ID NO: 404 - DNA, 25 bp, Homo sapiens
gctattgcccaagaattgctttcca

SEQ ID NO: 405 - DNA, 25 bp, Homo sapiens
gaattgctttccatgcacatattgt

SEQ ID NO: 406 - DNA, 25 bp, Homo sapiens
ttgtaaaaattccgctttgtgccac

SEQ ID NO: 407 - DNA, 25 bp, Homo sapiens
gctttgtgccacaggtcatgattgt

SEQ ID NO: 408 - DNA, 25 bp, Homo sapiens
agggactatttgtattgtatgttgc

SEQ ID NO: 409 - DNA, 25 bp, Homo sapiens
tcagggccaagttcgtggggacacc

SEQ ID NO: 410 - DNA, 25 bp, Homo sapiens
gaccaccttataccagcgttatgag

SEQ ID NO: 411 - DNA, 25 bp, Homo sapiens
ggttccaagccttaggggatgccgc

SEQ ID NO: 412 - DNA, 25 bp, Homo sapiens
ggagagtgtctgcggatacttccac

SEQ ID NO: 413 - DNA, 25 bp, Homo sapiens
gtcccacaaccgcagcgaggagttt

SEQ ID NO: 414 - DNA, 25 bp, Homo sapiens
aggatggactcttgcacatcactac

SEQ ID NO: 415 - DNA, 25 bp, Homo sapiens
acatcactacctgcagtttcgtggc

SEQ ID NO: 416 - DNA, 25 bp, Homo sapiens
ccctggaacagcctgagcttagctc

SEQ ID NO: 417 - DNA, 25 bp, Homo sapiens
tgcacagtgtttccctgtttatcca

SEQ ID NO: 418 - DNA, 25 bp, Homo sapiens
tatccatcccctgcaaactgcagag

SEQ ID NO: 419 - DNA, 25 bp, Homo sapiens
ggcactcattgcttgtggacggacc

(19) United States
(12) Patent Application Publication
(10) Pub. No.: US 2014/0162895 A1
(43) Pub. Date: Jun. 12, 2014

(54) SYSTEM, COMPUTER PROGRAM AND METHOD FOR DETERMINING BEHAVIOR OF THYROID TUMOR

(71) Applicant: SYSMEX CORPORATION, KOBE-SHI (JP)

(72) Inventors: Hiroyuki SHIMIZU, Kobe-shi (JP); Kazuki NAKABAYASHI, Kobe-shi (JP); Motonari DAITO, Kobe-shi (JP)

(73) Assignee: SYSMEX CORPORATION, KOBE-SHI (JP)

(21) Appl. No.: 14/092,467

(22) Filed: Nov. 27, 2013

(30) Foreign Application Priority Data
Nov. 29, 2012 (JP) 2012-261300
Nov. 5, 2013 (JP) 2013-229365

(51) Int. Cl. C12Q 1/68

(52) U.S. Cl. CPC C12Q 1/6886
USPC 506/9; 506/39; 506/16

(57) ABSTRACT

The invention provides a system adapted to a method for determining behavior of thyroid tumor. The system comprises a processor, and a memory, under control of said processor, including software instructions adapted to enable the system to perform operations.

### BACKGROUND OF THE INVENTION

1. Field of the Invention

The present invention relates to a system, a computer program, and a method for determining behavior of thyroid tumor.

2. Background

A disease in which nodules (tumors) are formed in the thyroid is generally referred to as “thyroid tumor”. Thyroid tumors are benign in many cases. However, among the cases, there is a case of a malignant tumor called “thyroid carcinoma”. In the diagnosis of thyroid tumors, the presence of thyroid tumors is confirmed by palpation, ultrasonography or the like. When the presence of thyroid tumors can be confirmed, in order to determine behavior (whether the tumors are benign or malignant), cells are collected from the tumors by fine-needle aspiration and cytological diagnosis is performed. However, the cytological diagnosis is performed based on the observation results of the cells and thus the determination results may vary depending on the experience and skill of a tester. Like the cases of follicular adenoma (benign tumor) and follicular carcinoma (malignant tumor), there are cases which have difficulty in being determined by even a skilled tester.

In the test for thyroid carcinoma, there is also a blood test for examining thyroid hormone or tumor markers in the blood. For example, it is known that when the thyroid carcinoma is medullary carcinoma, the value of calcitonin in thyroid hormone and the value of tumor marker CEA are increased. However, if the symptoms of thyroid carcinoma are shown, the value of the tumor marker is not always increased. Thus, thyroid carcinoma cannot be diagnosed only by the results of the blood test.

On the other hand, methods based on gene expression information have been recently studied and developed as new methods for determining behavior of thyroid tumor. For example, a microarray called Afirma (registered trademark) Thyroid FNA Analysis from Veracyte, Inc. is currently commercially available. Further, Alexander E K. et al. (Preoperative Diagnosis of Benign Thyroid Nodules with Indeterminate Cytology. NEng J Med, 2012; 367: 705-715)) discloses the results obtained by determining behavior of thyroid tumor using the Thyroid FNA Analysis. Specifically, as for specimens which are not determined to be either benign or malignant in cytodiagnosis (n=265), expression information of 142 genes is analyzed using Thyroid FNA Analysis to classify them into benignancy or suspicion of malignancy. Further, the classified results are compared with the results based on histopathological diagnosis. In the histopathological diagnosis, 85 specimens of 265 specimens are determined to be malignant. In the gene expression analysis, 78 specimens of the 85 specimens are determined to be suspicious of malignancy. That is, as the classification performance, the sensitivity is 92%, the specificity is 52%, the positive predictive value (PPV) is 47%, and the negative predictive value (NPV) is 93%. If performance when follicular neoplasms which are particularly difficult to determine (n=105) are classified into a follicular adenoma (benign) and follicular carcinoma (malignant) in the cytological diagnosis is calculated, the sensitivity is 95%, the specificity is 48%, the PPV is 30%, and the NPV is 98%.

Determining behavior of thyroid tumor based on the analysis results of the gene expression is an objective means and it is considered to be useful for the tumors which are difficult to determine. However, in the case of the determination using the currently commercially available Thyroid FNA Analysis, the specificity and PPV are very low as described above. On the other hand, the thyroid tumors are benign in many cases. Therefore, in the determination method in which the specificity and PPV are low, benign tumors in many subjects are mistakenly determined to be malignant in practice. As a result, there is a possibility of performing unnecessary medical treatment on the subjects with benign tumors.

### SUMMARY OF THE INVENTION

The scope of the present invention is defined solely by the appended claims, and is not affected to any degree by the statements within this summary.

The present inventors have exhaustively analyzed the expression levels of genes in diseased tissues with follicular adenoma and diseased tissues with follicular carcinoma. As a result, the present inventors have identified a gene cluster in which the expression is increased in benign and malignant tumors as a novel marker set. The present inventors have found that it is possible to determine whether thyroid tumor is a benign or malignant tumor based on the measured expression level of the marker set and it is possible to determine whether the malignant tumor is a tumor with poor prognosis, and have completed the present invention.

That is, the present invention provides a system adapted to a method for determining behavior of thyroid tumor. The system comprises a processor, and a memory, under control of said processor, including software instructions adapted to enable the system to perform operations. The operations comprise:

determining whether a thyroid tumor in a subject is benign or malignant based on the results of first, second, and third measurements;

wherein the expression levels of at least 6 genes selected from the group consisting of AIF1L, CDH16, FAM162B, FGFR2, GJB6, KCNJ13, KIAA1467, SLC25A15, TFCP2L1, TFF3, and TMEM171 in a sample collected from the subject are measured in the first measurement,

the expression levels of at least 7 genes selected from the group consisting of C4orf10, CCDC8, CD22, FAM125A, FAM174B, FBF1, GLB1L2, LOC644613, MAP2K2, MTG1, PFKL, PTDSS2, SF3A2, SLC2A11, VILL, VSIG2, and WDR18 in the sample are measured in the second measurement, and

the expression levels of at least 7 genes selected from the group consisting of ANXA1, C13orf33, CYP1B1, FAP, FN1, IL17RD, PDLIM4, RUNX2, and TIMP1 in the sample are measured in the third measurement.

Further, the present invention provides a computer program product for enabling a computer to determine behavior of thyroid tumor. The product comprises a computer readable medium, and software instructions, on the computer readable medium, for enabling the computer to perform predetermined operations. The operations comprises:

determining whether a thyroid tumor in a subject is benign or malignant based on the results of first, second, and third measurements, wherein the expression levels of at least 6 genes selected from the group consisting of AIF1L, CDH16, FAM162B, FGFR2, GJB6, KCNJ13, KIAA1467, SLC25A15, TFCP2L1, TFF3, and TMEM171 in a sample collected from the subject are measured in the first measurement,

the expression levels of at least 7 genes selected from the group consisting of C4orf10, CCDC8, CD22, FAM125A, FAM174B, FBF1, GLB1L2, LOC644613, MAP2K2, MTG1, PFKL, PTDSS2, SF3A2, SLC2A11, VILL, VSIG2, and WDR18 in the sample are measured in the second measurement, and the expression levels of at least 7 genes selected from the group consisting of ANXA1, C13orf33, CYP1B1, FAP, FN1, IL17RD, PDLIM4, RUNX2, and TIMP1 in the sample are measured in the third measurement.

Further, the present invention provides a method for determining behavior of thyroid tumor. The method comprises:

applying a sample collected from a subject with a thyroid tumor to a microarray capable of measuring the expression levels of the following genes (a) to (c):

(a) at least 6 genes selected from the group consisting of AIF1L, CDH16, FAM162B, FGFR2, GJB6, KCNJ13, KIAA1467, SLC25A15, TFCP2L1, TFF3, and TMEM171;

(b) at least 7 genes selected from the group consisting of C4orf10, CCDC8, CD22, FAM125A, FAM174B, FBF1, GLB1L2, LOC644613, MAP2K2, MTG1, PFKL, PTDSS2, SF3A2, SLC2A11, VILL, VSIG2, and WDR18; and (c) at least 7 genes selected from the group consisting of ANXA1, C13orf33, CYP1B1, FAP, FN1, IL17RD, PDLIM4, RUNX2, and TIMP1;

detecting optical information about the expression levels of the genes (a) to (c) obtained from the microarray using a microarray scanner after the applying step;

sending the optical information detected in the detecting step to a CPU of a computer connected to the microarray scanner;

calculating the expression levels of the genes (a) to (c), based on the optical information by the CPU; and

determining whether the thyroid tumor in the subject is benign or malignant using the values calculated by the CPU in the calculating step.

### BRIEF DESCRIPTION OF THE DRAWINGS

FIG. 1 shows the results of cluster analysis of the expression levels of 37 genes obtained by measuring specimens with follicular neoplasm (Learning set) with a microarray;

FIG. 2 shows the results of cluster analysis of the expression levels of 37 genes obtained by measuring specimens with follicular neoplasm (Validation set) with a microarray;

FIG. 3 shows the results of cluster analysis of the expression levels of 37 genes obtained by measuring specimens with thyroid tumors other than follicular neoplasm with a microarray;

FIG. 4 shows a determination system of this embodiment;

FIG. 5 is a block diagram showing a functional configuration of the determination system shown in FIG. 4;

FIG. 6 is a block diagram showing a hardware configuration of the determination system shown in FIG. 4; and

FIG. 7 is a flow chart of the determination using the determination system shown in FIG. 4.

FIG. 8 is a table showing the determination according to the present invention.

### DETAILED DESCRIPTION OF THE PREFERRED EMBODIMENTS

The preferred embodiments of the present invention will be described hereinafter with reference to the drawings.

In the determination method of the present invention, as for biological samples collected from subjects with thyroid tumors, the expression levels of at least 6 genes selected from a first gene cluster, the expression levels of at least 7 genes selected from a second gene cluster, and the expression levels of at least 7 genes selected from a third gene cluster are measured.

The term “thyroid tumors” used herein means nodules (tumors) formed in the thyroid and intends to include both benign and malignant tumors. In the art, the thyroid tumors are also referred to as “thyroid nodules” or “adenomatous goiter”.

The term “determining behavior of thyroid tumor” means determining whether the thyroid tumor is benign or malignant. When the thyroid tumor is malignant, it can include determining whether the tumor is a malignant tumor with poor prognosis or a malignant tumor with good prognosis.

Here, the term “malignant tumor with poor prognosis” means a malignant tumor which has a high degree of malignancy and a high possibility of recurrence and/or metastasis among the malignant thyroid tumors and thus has a high mortality rate in patients. Further, the term “malignant tumor with good prognosis” means a malignant tumor which has a low degree of malignancy and a low possibility of recurrence and/or metastasis among the malignant thyroid tumors and thus has a low mortality rate in patients. The malignant tumor with poor prognosis largely differs in therapeutic strategies from the malignant tumor with good prognosis. Consequently, distinction between the malignant tumor with poor prognosis and the malignant tumor with good prognosis becomes useful information in determination of therapeutic strategies.

The term “subjects with thyroid tumors” used herein means subjects who are confirmed to have thyroid tumors or strongly suspected to have thyroid tumors by palpation or ultrasonography.

In this embodiment, the biological sample is not particularly limited as long as it is a sample derived from a living body which contains cells derived from the thyroid of the subject. Particularly, a sample derived from a living body which contains cells derived from the lesion site in the thyroid is preferred. Examples of the sample derived from a living body include blood, serum, plasma, lymph, cells collected by fine-needle aspiration, and tissues collected by surgery. Further, cultures obtained by culturing the cells and tissues extracted from subjects may also be used as a biological sample.

The term “gene cluster” used herein means a group consisting of plural genes.

The term “gene expression level” used herein means an amount of RNA transcribed from each gene or an amount of material reflecting the amount. Therefore, in the embodiment of the present invention, a nucleic acid (particularly RNA) is extracted from a biological sample. The amount of RNA derived from each gene contained in the nucleic acid or the amount of cRNA obtained by IVT-amplification of cDNA or cDNA obtained by reverse transcription of the RNA is measured as the gene expression level. The amount of RNA transcribed from each gene is small in many cases. Accordingly, it is preferable to measure the amount of cDNA obtained from the RNA or the amount of cRNA.

A method for extracting a nucleic acid including RNA from a biological sample is known in the art. RNA can be extracted from a biological sample by, for example, a method comprising mixing the biological sample with a treatment solution containing a surfactant which solubilizes cells or tissues (sodium cholate, sodium dodecyl sulfate etc.), physically treating (agitation, homogenization, ultrasonication etc.) the resulting mixture to release RNA contained in the biological sample into the mixed solution. Preferably, the mixed solution containing the released RNA is centrifuged to recover a supernatant, and the supernatant is extracted with phenol/chloroform to purify the RNA. The extraction and purification of RNA from the biological sample can be performed using a commercially available kit.

In the art, the base sequences themselves of the respective genes are known. These base sequences can be obtained from, for example, a known database (http://www.ncbi.nlm.nih.gov/ (provided by National Center for Biotechnology Information (NCBI)). The NCBI Accession Nos and Probe IDs of the genes, and Seq ID Nos. of oligonucleotide probes included in each probe set are shown in Table 1. The IDs are up to date as of Nov. 16, 2012. Probe ID is a number for identifying probes on GeneChip (registered trademark) Human Genome U133 Plus 2.0 Array (Affymetrix, Inc.). The base sequences of probes can be obtained from, for example, the website of Affymetrix, Inc. (http://www.affymetrix.com/analysis/index.affx).

TABLE 1

| | | | Seq ID |
|---|---|---|---|
| Symbol | Gene IDs (NCBI) | Probe Set ID | NOs |
| AIF1L | NM_031426 | 223075_s_at | 1-11 |
| CDH16 | NM_004062 | 206517_at | 12-22 |
| FAM162B | NM_001085480 | 228875_at | 23-33 |
| FGFR2 | NM_000141, | 203638_s_at | 34-44 |
| | NM_001144913, | | |
| | NM_001144914, | | |
| | NM_001144915, | | |
| | NM_001144916, | | |
| | NM_001144917, | | |
| | NM_001144918, | | |
| | NM_001144919, | | |
| | NM_022970 | | |
| GJB6 | NM_001110219, | 231771_at | 45-55 |
| | NM_001110220, | | |
| | NM_001110221, | | |
| | NM_006783 | | |
| KCNJ13 | NM_001172416, | 210179_at | 56-66 |
| | NM_001172417, | | |
| | NM_002242 | | |
| KIAA1467 | NM_020853 | 213234_at | 67-77 |
| SLC25A15 | NM_014252 | 218653_at | 78-88 |
| TFCP2L1 | NM_014553 | 227642_at | 89-99 |
| TFF3 | NM_003226 | 204623_at | 100-110 |
| TMEM171 | NM_001161342.1, | 240770_at | 111-121 |
| | NM_173490 | | |
| C4orf10 | NR_015453 | 214123_s_at | 122-132 |
| CCDC8 | NM_032040 | 223495_at | 133-143 |
| CD22 | NM_001771 | 38521_at | 144-159 |
| FAM125A | NM_138401 | 227864_s_at | 160-170 |
| FAM174B | NM_207446 | 51158_at | 171-186 |
| FBF1 | NM_001080542 | 1555288_s_at | 187-197 |
| GLB1L2 | NM_138342 | 213713_s_at | 198-208 |
| LOC644613 | BC025792 | 1563426_a_at | 209-219 |
| MAP2K2 | NM_030662 | 202424_at | 220-230 |
| MTG1 | NM_138384 | 212767_at | 231-241 |
| PFKL | NM_002626, NR_024108 | 211065_x_at | 242-252 |
| PTDSS2 | NM_030783 | 221005_s_at | 253-263 |
| SF3A2 | NM_007165 | 37462_i_at | 264-276 |
| SLC2A11 | NM_001024938, | 232167_at | 277-287 |
| | NM_001024939, | | |
| | NM_030807 | | |
| VILL | NM_015873 | 209950_s_at | 288-298 |
| VSIG2 | NM_014312 | 228232_s_at | 299-309 |
| WDR18 | NM_024100 | 209461_x_at | 310-320 |
| ANXA1 | NM_000700 | 201012_at | 321-331 |
| C13orf33 | NM_032849 | 227058_at | 332-342 |
| CYP1B1 | NM_000104 | 202437_s_at | 343-353 |
| FAP | NM_004460 | 209955_s_at | 354-364 |
| FN1 | NM_002026, NM_054034, | 211719_x_at | 365-375 |
| | NM_212474, NM_212475, | | |
| | NM_212476, NM_212478, | | |
| | NM_212482 | | |
| IL17RD | NM_017563 | 227997_at | 376-386 |
| PDLIM4 | NM_001131027, NM_003687 | 214175_x_at | 387-397 |
| RUNX2 | NM_001015051, | 232231_at | 398-408 |
| | NM_001024630, | | |
| | NM_004348 | | |
| TIMP1 | NM_003254 | 201666_at | 409-419 |

The 37 genes are a cluster of genes in which the fact that the expression in cases with benign and malignant tumors is increased is found out by the present inventors. Specifically, the first gene cluster is a cluster of genes in which an increase in the expression of the cases with benign tumors is more than that of the cases with malignant tumors. In addition, the second and third gene clusters are a cluster of genes in which an increase in the expression of the cases with malignant tumors is more than that of the cases with benign tumors. Further, the present inventors have found that the expression of the second gene cluster is increased, particularly in the cases of malignant tumors with poor prognosis, and the expression of the third gene cluster is increased, particularly in the cases of malignant tumors with good prognosis.

In the embodiment of the present invention, there is no particular restriction to measure the expression level of any of the genes of the first, second, and third gene clusters, and it can be arbitrarily selected. As an example, a method for measuring the expression level of at least 6 genes: AIF1L, FAM162B, FGFR2, GJB6, KIAA1467, and TFF3 of the first gene cluster, the expression level of at least 7 genes: FAM174B, MAP2K2, MTG1, PFKL, PTDSS2, SF3A2, and WDR18 of the second gene cluster, and the expression level of at least 7 genes: ANXA1, CYP1B1, FAP, IL17RD, PDLIM4, RUNX2, and TIMP1 of the third gene cluster is listed. However, the determination method of the present invention is not limited to the example.

In the embodiment of the present invention, the method for measuring the gene expression level is not particularly limited and it can be selected from any known method in the art. A measurement method using a microarray or a nucleic acid amplification method is preferred.

The microarray used for the determination method of the present invention is not particularly limited as long as it is a chip in which a nucleic acid probe having about 20 to 25 nucleotides which can specifically hybridize to cDNA or cRNA derived from RNAs transcribed from the genes or the RNAs (hereinafter, also referred to as “target nucleic acid molecule”) is immobilized on an appropriate substrate. The probe can be appropriately designed based on the base sequences of the genes. Further, the microarray can be produced by any known method in the art. In the embodiment of the present invention, a commercially available microarray may be used as long as the probe which can specifically hybridize to the target nucleic acid molecule is mounted. Examples of the microarray include GeneChip (registered trademark, manufactured by Affymetrix, Inc.).

As used herein, the term “can specifically hybridize” means that a probe can hybridize to a target nucleic acid molecule under a stringent condition. The term “stringent condition” means a condition under which the probe can hybridize to the target nucleic acid molecule with a detectably higher extent than it does to a nucleic acid molecule other than the target nucleic acid molecule (e.g. more than at least two times of the background). The stringent condition generally depends on the sequences and varies depending on various circumstances. Generally, the stringent condition is selected so that it is about 5° C. lower than a thermal melting point (Tm) of the specific sequence under a certain ionic strength and pH. This Tm is a temperature at which 50% of the complementary probe hybridizes to the base sequence of the target nucleic acid molecule in equilibrium (under a certain ionic strength, pH and nucleic acid composition). Such condition may be those which are used in hybridization techniques between polynucleotides known in the art. Specifically, it may be a condition of pH 7.0 to 9.0, a salt concentration of lower than about 1.5 M Na-ion, more specifically about 0.01 to 1.0 M Na-ion concentration (or other salt), and a temperature of at least about 30° C. For example, the stringent condition in a microarray technique includes the hybridization at 37° C. in 50% formamide, 1 M NaCl, and 1% SDS; and washing at 60 to 65° C. in 0.1×SSC.

When the microarray technique is used, the target nucleic acid molecule is preferably labeled with a labeling substance known in the art. Labeling of the target nucleic acid molecule facilitates measurement of a signal from a probe on a microarray. In the embodiment of the present invention, RNA extracted from a biological sample may be labeled. Preferably, cDNA or cRNA derived from the RNA is labeled. Examples of the labeling substance include fluorescent substances, haptens such as biotin, and radioactive substances. Examples of the fluorescent substances include Cy3, Cy5, FITC, Alexa Fluor (trademark). A method for labeling RNA, cDNA, and cRNA with these labeling substances is known in the art.

In the measurement using a microarray, the gene expression level is obtained as a signal from a probe, such as fluorescence intensity, luminescence intensity or amount of current. Each of the signals may be detected using a scanner installed in a general microarray analyzer. Examples of the scanner include GeneChip (registered trademark) Scanner 3000 7G (Affymetrix, Inc.) and Illumina (registered trademark) Bead Array Reader (Illumina).

Examples of the nucleic acid amplification method suitable for the determination method of the present invention include a polymerase-chain-reaction (PCR) assay, a strand displacement activity assay, a ligase-chain-reaction assay, and a transcriptional amplification method. These nucleic acid amplification methods themselves are known in the art. As the PCR assay, for example, a real-time RT-PCR assay is listed. As the strand displacement activity assay, for example, a real-time RT-LAMP assay (refer to, for example, U.S. Pat. No. 6,410,278) is listed. As the transcriptional amplification method, for example, a TAS assay is listed. Among these methods, the real-time RT-PCR assay and the real-time RT-LAMP assay are preferred.

The primer to be used for a nucleic acid amplification method is not particularly limited as long as it is a primer which can be amplified by specific hybridization to the target nucleic acid molecule. The stringent condition in the PCR assay may be a condition of pH 7.0 to 9.0, 0.01 to 0.1 M Tris HCl, 0.05 to 0.15 M K-ion concentration (or other salt), and a temperature of at least about 55° C. Such a primer can be appropriately designed based on the base sequences of the respective genes. Further, the primer is preferably designed according to the type of the nucleic acid amplification method. The length of the primer is usually from 5 to 50 nucleotides, preferably from 10 to 40 nucleotides. The primer can be produced by any nucleic acid synthesis method known in the art.

The primer may be labeled with any labeling substance known in the art. Labeling of the primer may be performed using radioactive elements or non-radioactive molecules. Examples of the radioactive isotope include ³²P, ³³P, ³⁵S, ³H, and ¹²⁵I. Examples of the non-radioactive substance include ligands such as biotin, avidin, streptoavidin, and digoxigenin; hapten, pigment, and luminescent reagents such as chemiluminescent, bioluminescent, fluorescent and phosphorescent reagents.

The reaction condition of the nucleic acid amplification method varies depending on the type of the nucleic acid amplification method and the base sequences of the primer, and it can be appropriately set with reference to, for example, a method described in Molecular Cloning: A Laboratory Manual (2nd ed.) (Sambrook, J. et al. Cold Spring Harbor Laboratory Press, New York (1989)).

The gene expression level is measured as described above, followed by determining whether the thyroid tumor in the subject with thyroid tumor is benign or malignant based on the measured expression levels of genes of the first, second, and third gene clusters in the determination method of the present invention. Here, the benign tumor is not particularly limited as long as it is a thyroid tumor known to be benign in the art, and it preferably means at least one of follicular adenoma, adenomatous nodules, and cysts. Further, the malignant tumor is not particularly limited as long as it is a thyroid tumor known to be malignant in the art, and it preferably means at least one of follicular carcinoma, papillary carcinoma, poorly-differentiated carcinoma, and undifferentiated carcinoma. The malignant tumor includes a malignant tumor with poor prognosis and a malignant tumor with good prognosis.

In the embodiment of the present invention, it is preferable that the expression states of the gene clusters are compared based on the measured expression levels of genes of the first, second, and third gene clusters and the determination is performed based on the results. That is, when the expression state of the genes measured in the first measurement is more than or equal to the expression state of the genes measured in the second measurement, and when the expression state of the genes measured in the first measurement is more than or equal to the expression state of the genes measured in the third measurements, the thyroid tumor of the subject is determined to be benign. On the contrary, when the expression state of the genes measured in either the second or third measurement is more than the expression state of the genes measured in the first measurement, the thyroid tumor of the subject is determined to be malignant.

In the present invention, the term “expression state of genes selected from the gene cluster” means an indicator of the level or tendency of the gene expression in a plurality of genes selected from predetermined gene clusters. As the expression state, for example, a measure of central tendency of the expression levels (measured value) in a plurality of selected genes is listed. The measure of central tendency is not particularly limited as long as it is a statistics value which summarizes the distribution of the gene expression in a plurality of genes selected from predetermined gene clusters. Examples of the measure of central tendency include an average, a median, a maximum, and a mode. Among them, the average is particularly preferred. The term “the expression state is increased” means a measure of central tendency of the expression levels in a plurality of genes selected from certain gene clusters is higher than that of the expression levels in a plurality of genes selected from the remaining gene clusters. Therefore, when an average of the expression levels is used as an expression state of genes selected from a gene cluster, comparison of the expression states is performed as follows. As for biological samples of subjects, the sum of the expression levels of at least 6 genes selected from the first gene cluster is calculated. The calculated value is divided by the number of the selected genes to obtain an average. The same calculation is performed on at least 7 genes selected from the second and third gene clusters. Then, the obtained three averages are compared. For example, when the average in the genes selected from the first gene cluster is the highest, the thyroid tumor of the subject is determined to be benign. On the contrary, when the average in the genes selected from the second or third gene cluster is the highest, the thyroid tumor of the subject is determined to be malignant.

In the embodiment of the present invention, the expression state of genes selected from each of the gene clusters in biological samples of subjects may be obtained by using the expression level data previously obtained as for the cases with benign and malignant tumors in which behavior is determined by pathological diagnosis (control samples). For example, the multivariate analysis is performed using the expression level data previously obtained as for the control samples so that a formula which reflects or predicts the expression states of genes selected from the gene clusters can be obtained. The information on the expression states of the gene clusters in the biological samples of the subjects is obtained by using the obtained formula. Based on the information, the samples can be determined. Preferable examples of the multivariate analysis include discriminant analysis. Examples of the discriminant analysis include Fischer's linear discriminant analysis, Mahalanobis distance-based discriminant analysis, Euclid distance-based discriminant analysis, Multi-group discriminant analysis, variable selection, and canonical discriminant analysis. Among them, particularly the Euclid distance-based discriminant analysis is preferred. The Euclid distance shows similarity in gene expression patterns among the control samples and the biological samples of the subjects. The Euclid distance can be obtained as follows. Based on the expression level data of the control samples which is previously obtained, the control samples are first classified into three groups by the cluster analysis known in the art. Conveniently the three groups are referred to as “Cluster 1” with predominant expression of genes selected from the first gene cluster, “Cluster 2” with predominant expression of genes selected from the second gene cluster, and “Cluster 3” with predominant expression of genes selected from the third gene cluster, respectively. Subsequently, the data of the expression levels of the groups is plotted to calculate an average of the groups. Then, as for the average of the groups and the expression level data obtained from the biological samples of the subjects, Euclid distances among the control samples and the biological samples of the subjects are calculated.

When the Euclid distance is used, the term “expression state is increased” means that a Euclid distance between data of a certain cluster and the data of the subject is closer than an Euclid distance between data of the remaining clusters and the data of the subject. Therefore, comparison of the expression states is performed as follows. Three Euclid distances calculated are compared. For example, when the distance from Cluster 1 is the closest, the thyroid tumor of the subject is determined to be benign. On the contrary, when the distance from Cluster 2 or Cluster 3 is the closest, the thyroid tumor of the subject is determined to be malignant.

In another embodiment of the present invention, the expression states of genes selected from the gene clusters are compared based on the measured expression levels of genes of the first, second, and third gene clusters. Based on the results, behavior of thyroid tumor in a subject can be determined as follows.

That is, when the expression state of the genes measured in the first measurement is more than or equal to the expression state of the genes measured in the second measurement, and when the expression state of the genes measured in the first measurement is more than or equal to the expression state of the genes measured in the third measurement, the thyroid tumor of the subject is determined to be benign.

When the expression state of the genes measured in the second measurement is more than the genes measured in the first measurement, and when the expression state of the genes measured in the second measurement is more than or equal to the expression state of the genes measured in the third measurement, the thyroid tumor of the subject is determined to a first malignant tumor.

When the expression state of the genes measured in the third measurement is more than the expression states of the genes measured in the first and second measurements, the thyroid tumor of the subject is determined to a second malignant tumor.

The term “first malignant tumor” used herein means a malignant tumor with poor prognosis and the term “second malignant tumor” means a malignant tumor with good prognosis. The malignant tumor with poor prognosis is not particularly limited as long as it is a malignant thyroid tumor which is known to be poor prognosis in the art. Preferable examples thereof include at least one of undifferentiated carcinoma, poorly-differentiated carcinoma, and follicular carcinoma with widely invasive and/or vascular invasion. The malignant tumor with poor prognosis is not particularly limited as long as it is a malignant thyroid tumor which is known to be poor prognosis in the art. Preferable examples thereof include at least one of papillary carcinoma and follicular carcinoma without widely invasive and vascular invasion.

A marker set for determining behavior of thyroid tumor (hereinafter also referred to as “marker set”) is included in the scope of the present invention. The marker set of the present invention includes at least 6 genes selected from the first gene cluster, at least 7 genes selected from the second gene cluster, and at least 7 genes selected from the third gene cluster.

In the embodiment of the present invention, there is no particular restriction to use any of the genes of the first, second, and third gene clusters as a marker, and it can be arbitrarily selected. As an example, a marker set including at least 6 genes: AIF1L, FAM162B, FGFR2, GJB6, KIAA1467, and TFF3 of the first gene cluster, at least 7 genes: FAM174B, MAP2K2, MTG1, PFKL, PTDSS2, SF3A2, and WDR18 of the second gene cluster, and at least 7 genes: ANXA1, CYP1B1, FAP, IL17RD, PDLIM4, RUNX2, and TIMP1 of the third gene cluster is listed. However, the marker set of the present invention is not limited to the example.

In the embodiment of the present invention, the expression levels of genes in a sample containing RNAs prepared from the biological sample collected from the subject is analyzed and the behavior of thyroid tumor in the subject can be determined based on the obtained expression levels. The measurement and analysis of the expression levels of the markers are the same as described above.

A kit for determining behavior of thyroid tumor (hereinafter, also referred to as “kit of the present invention”) is included in the scope of the present invention. That is, the present invention provides the kit for determining behavior of thyroid tumor including a probe set or primer set for measuring the expression levels of at least 6 genes selected from the first gene cluster composed of AIF1L, CDH16, FAM162B, FGFR2, GJB6, KCNJ13, KIAA1467, SLC25A15, TFCP2L1, TFF3, and TMEM171, the expression levels of at least 7 genes selected from the second gene cluster composed of C4orf10, CCDC8, CD22, FAM125A, FAM174B, FBF1, GLB1L2, LOC644613, MAP2K2, MTG1, PFKL, PTDSS2, SF3A2, SLC2A11, VILL, VSIG2, and WDR18, and the expression levels of at least 7 genes selected from the third gene cluster composed of ANXA1, C13orf33, CYP1B1, FAP, FN1, IL17RD, PDLIM4, RUNX2, and TIMP1.

The kit of the present invention can be suitably used for the determination method of the present invention. The probe and primer sets included in the kit of the present invention are the same described in the probe or primer which can specifically hybridize to the target nucleic acid molecule. The kit of the present invention may be in the form of microarray in which the probe is immobilized on an appropriate substrate.

In the embodiment of the present invention, there is no particular restriction to include any of the probes or primer for measuring the gene expression in the kit, and it can be arbitrarily selected. As an example, a kit including a probe set or primer set for measuring at least the expression levels of AIF1L, FAM162B, FGFR2, GJB6, KIAA1467, and TFF3 of the first gene cluster, at least the expression levels of FAM174B, MAP2K2, MTG1, PFKL, PTDSS2, SF3A2, and WDR18 of the second gene cluster, and at least the expression levels of ANXA1, CYP1B1, FAP, IL17RD, PDLIM4, RUNX2, and TIMP1 of the third gene cluster is listed. However, the kit of the present invention is not limited to the example.

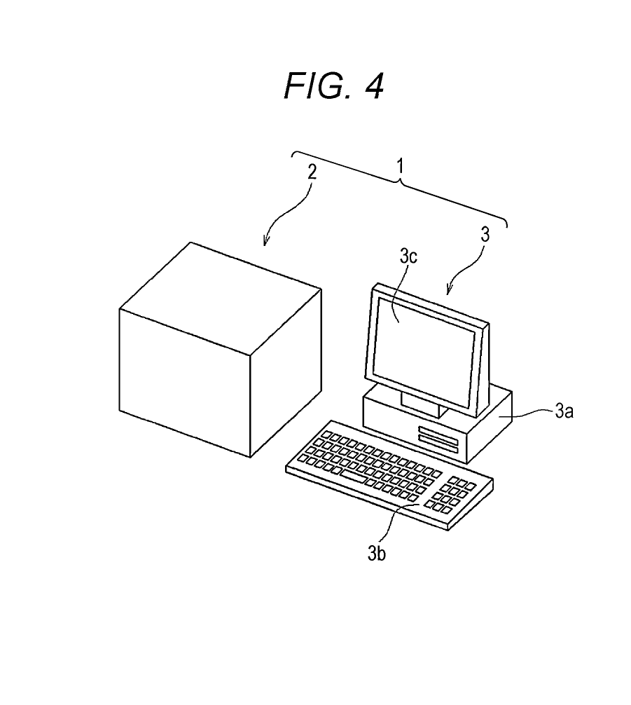

The determination of behavior of thyroid tumor can be performed by, for example, the determination system 1 shown in FIG. 4. Hereinafter, the present invention will be more specifically described with reference to the attached drawings; however, the present invention is not limited to the embodiment.

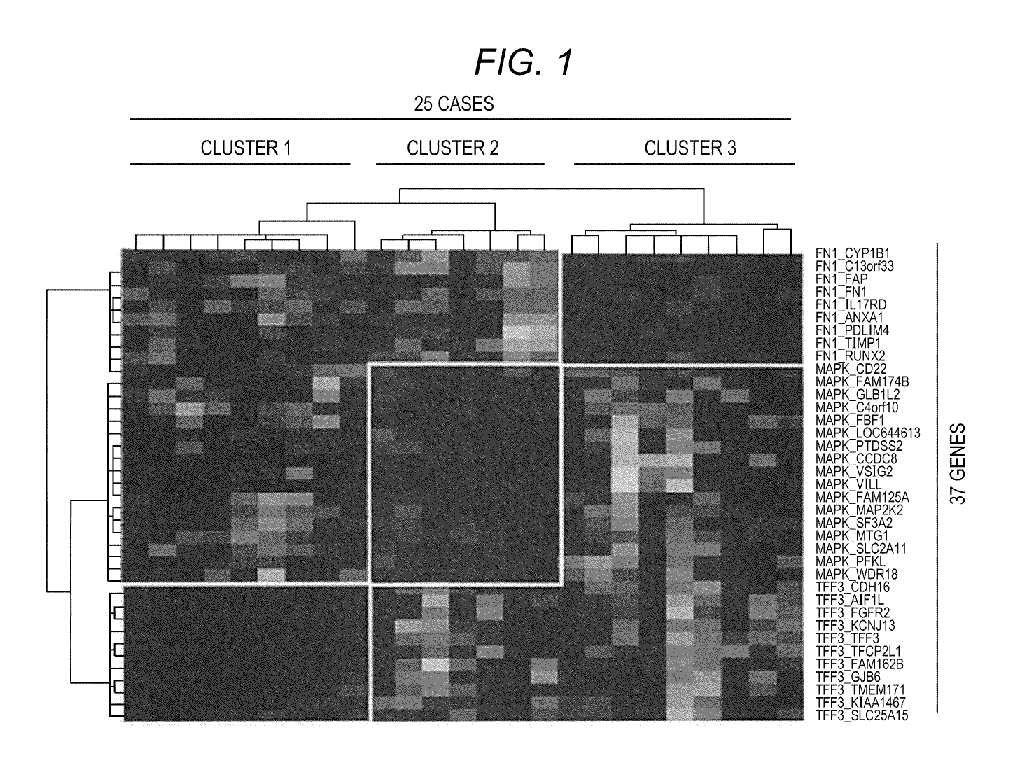

FIG. 1 is an outline explanatory view of a system according to one embodiment of the present invention. The determination system 1 shown in FIG. 1 includes a measurement device 2 and a computer system 3 connected to the measurement device 2.

In this embodiment, the measurement device 2 is a microarray scanner that detects a signal based on the hybridization of the probe and the target nucleic acid molecule on a microarray. The measurement device 2 detects signals corresponding to expression levels of genes selected from a first gene cluster, a second gene cluster, and a third gene cluster. Specifically, in this embodiment, the signals represent optical information. As the optical information, for example, a fluorescent signal is listed; however, the present invention is not limited only thereto. In this case, when the microarray after contact with the target nucleic acid molecule is set in the measurement device 2, the measurement device 2 obtains optical information based on the target nucleic acid molecule bound to the probe on the microarray and sends the obtained optical information to the computer system 3.

Any microarray scanner may be used as long as it can detect a signal based on the target nucleic acid molecule. The signal varies depending on the labeling substance used for labeling cDNA or cRNA in the measurement sample. Thus, as the microarray scanner, one suitable for detecting the signal from the labeling substance can be appropriately selected according to the type of the labeling substance. For example, when the labeling substance is a radioactive substance, a microarray scanner which can detect radioactive rays from the radioactive substance can be used as the measurement device 2.

When the gene expression level is detected by the nucleic acid amplification method, the measurement device 2 may be a nucleic acid amplification detector. In this case, a reaction solution containing DNA from a biological sample, an enzyme for nucleic acid amplification, and a primer is set in the measurement device 2. The nucleic acid in the reaction solution is amplified by the nucleic acid amplification method. The measurement device 2 obtains optical information such as fluorescence generated from the reaction solution by the amplification reaction or turbidity of the reaction solution and sends the optical information to the computer system 3.

The computer system 3 includes a computer main body 3a, an input device 3b, and a display unit 3c that displays specimen information, determination results, and the like. The computer system 3 receives optical information from the measurement device 2. The processor of the computer system 3 executes a program that determines behavior of thyroid tumor based on the optical information.

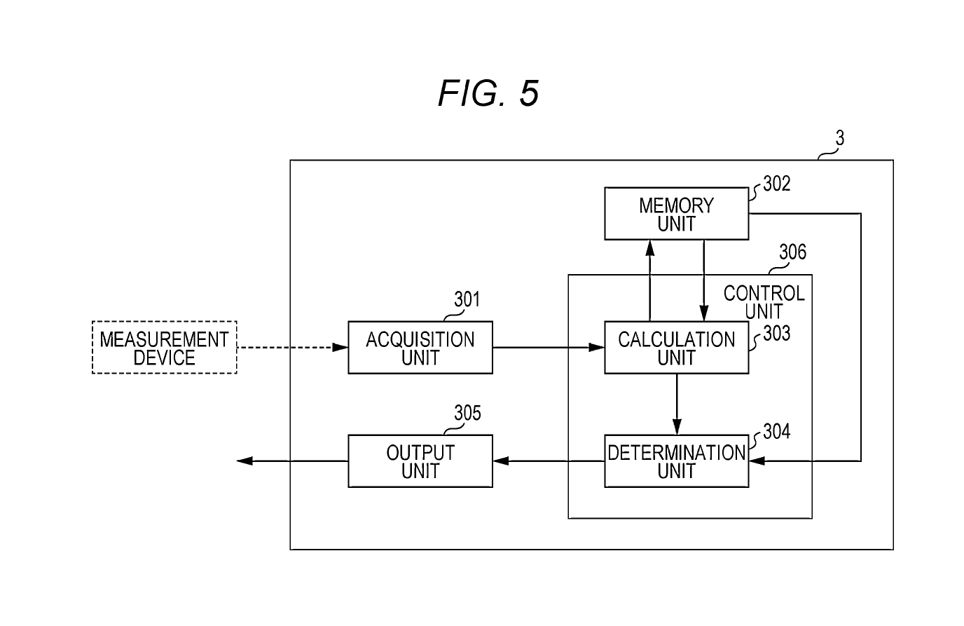

FIG. 5 is a block diagram showing a functional configuration of the determination system shown in FIG. 4.

The computer system 3 comprises an acquisition unit 301, a memory unit 302, a calculation unit 303, a determination unit 304, and an output unit 305 as shown in FIG. 5. The acquisition unit 301 is communicably connected to the measurement device 2 via a network. The calculation unit 303 and the determination unit 304 form a control unit 306.

The acquisition unit 301 obtains information sent from the measurement device 2.

The memory unit 302 stores a program that distinguishes behavior of thyroid tumor.

The calculation unit 303 calculates fluorescence intensity etc. using information obtained by the acquisition unit 301 and analyzes the gene expression state. As described above, as the gene expression state, for example, a measure of central tendency of the expression levels (measured values) in a plurality of selected genes is listed.

The determination unit 304 determines behavior of thyroid tumor based on the gene expression state analyzed by the calculation unit 303 and determination criteria stored in the memory unit 302.

The output unit 305 outputs determination results by the determination unit 304.

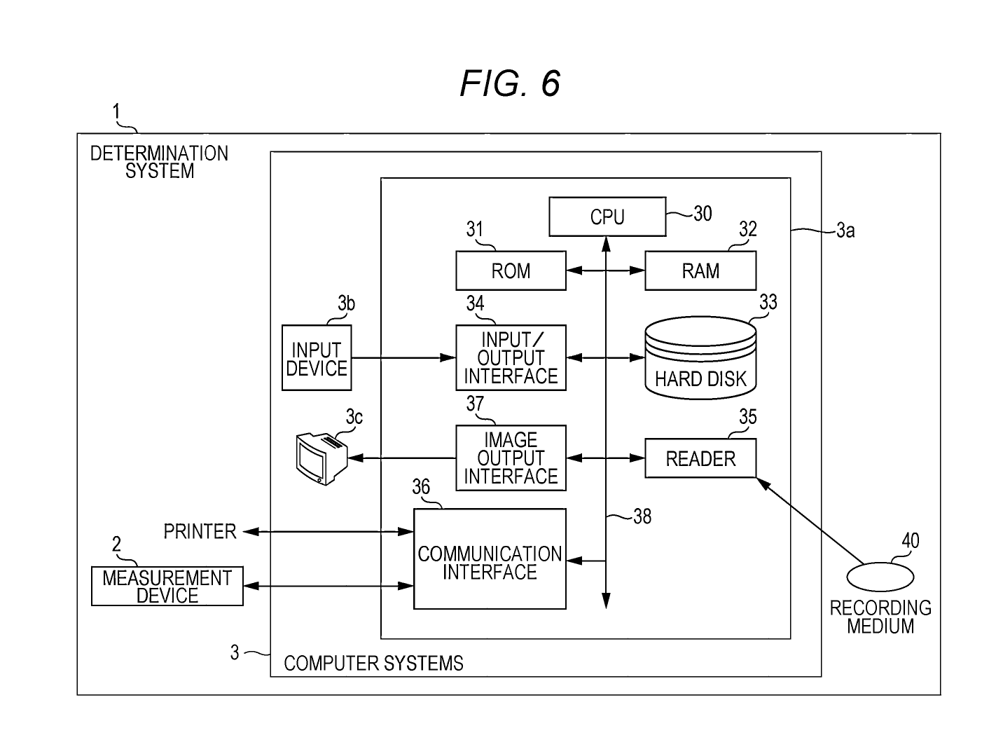

FIG. 6 is a block diagram showing a hardware configuration of the determination system shown in FIG. 4.

As shown in FIG. 6, the computer main body 3a comprises a CPU (Central Processing Unit) 30, a ROM (Read Only Memory) 121, a ROM 32, a hard disk 33, an input/output interface 34, a read-out device 35, a communication interface 36, and an image output interface 37. The CPU 30, the ROM 31, the RAM (Random Access Memory) 32, the hard disk 33, the input/output interface 34, the read-out device 35, the communication interface 36, and the image output interface 37 are data-communicably connected by a bus 38.

The CPU 30 is capable of executing the computer programs stored in the ROM 31 and the computer programs loaded in the ROM 32. The CPU 30 executes an application program to realize the blocking of the functions. Thus, the computer system functions as a terminal of a system for determining behavior of thyroid tumor.

The ROM 31 is configured by mask ROM, PROM, EPROM, EEPROM, and the like. Computer programs to be executed by the CPU 30 and data used for the same are recorded in the ROM 31.

The ROM 32 is configured by SRAM, DRAM, and the like. The ROM 32 is used to read out the computer programs recorded on the ROM 31 and the hard disc 33. In executing the computer program, the ROM 32 is used as a work region of the CPU 30.

The hard disk 33 is installed with an operating system to be executed by the CPU 30, a computer program such as an application program (computer program for determining the presence or absence of sensitivity to preoperative chemotherapy on breast cancer), and data used to execute the computer program.

The read-out device 35 is configured by a flexible disk drive, a CD-ROM drive, a DVD-ROM drive, and the like. The read-out device 35 is able to read out computer programs or data recorded on a portable recording medium 40.

The input/output interface 34 includes a serial interface such as USB, IEEE1394, and RS-232C; a parallel interface such as SCSI, IDE, and IEEE1284; and an analog interface such as D/A converter and A/D converter. The input/output interface 34 is connected to the input device 3b such as a keyboard and a mouse. Operators can use the input device 3b to input data into the computer main body 3a.

The communication interface 36 is, for example, an Ethernet (registered trademark) interface. The computer system 3 can receive optical information from the measurement device 2 through the communication interface 36 and send print data to a printer.

The image output interface 37 is connected to the display unit 3c configured by LCD, CRT, or the like. Thus, the display unit 3c can output an image signal corresponding to the image data provided from the CPU 30. The display unit 3c displays an image (screen) according to the input image signal.

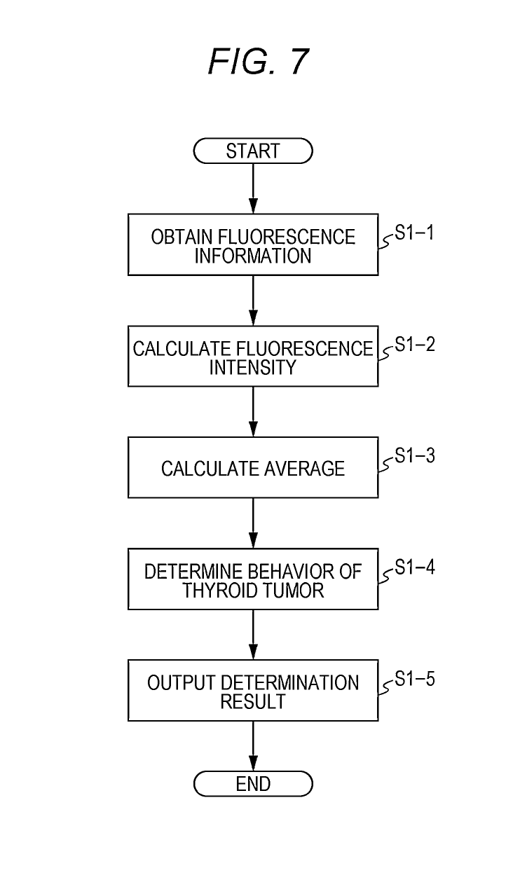

Subsequently, the determination procedure by the determination system 1 will be described. FIG. 7 is a flow chart of the determination using the determination system shown in FIG. 4. Here, the present invention will be described using fluorescence information based on a nucleic acid being measured which is bound to a probe on a microarray and taking an example of the case where averages as “the expression states of genes selected from the gene clusters” are used to perform determination. However, the present invention is not limited only to the embodiment.

In step S1-1, the acquisition unit 301 of the determination system 1 first obtains fluorescence information from the measurement device 2. In step S1-2, the calculation unit 303 calculates fluorescence intensity from the fluorescence information obtained by the acquisition unit 301 and sends it to the memory unit 302.

Subsequently, in step S1-3, the calculation unit 303 calculates an average of the expression levels of a plurality of selected genes based on the fluorescence intensity stored in the memory unit 302.

Thereafter, in step S1-4, the determination unit 304 determines behavior of thyroid tumor using an average calculated by the calculation unit 303 and determination criteria stored in the memory unit 302. The determination results are sent to the output unit 305.

Thereafter, in step S1-5, the output unit 305 outputs the determination results, allows the display unit 3c to display them, and allows the printer to print them.

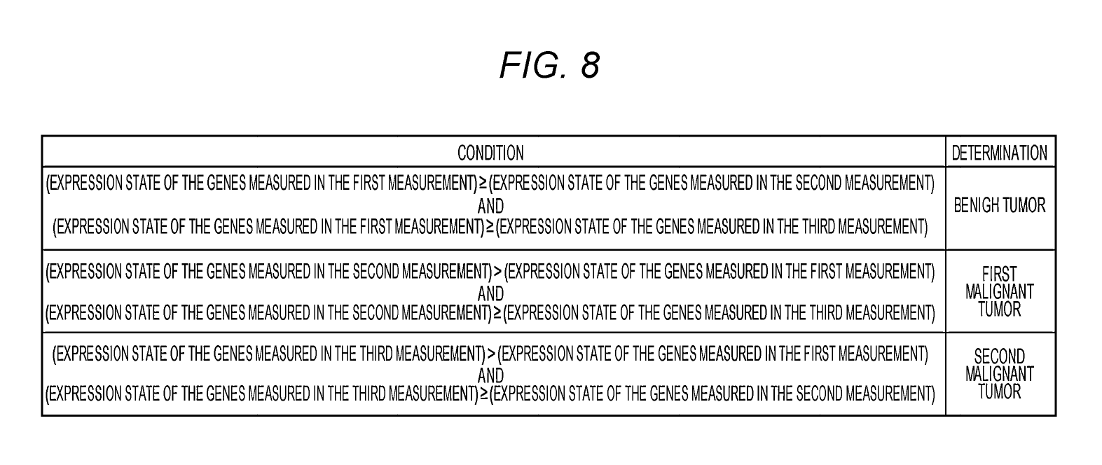

FIG. 8 is a view showing the method for determining behavior of thyroid tumor according to the determination of the present invention. When the conditions shown in the left column of FIG. 8 are satisfied, determination results shown in the right column of FIG. 8 are obtained. In this regard, an inequality sign “>” in FIG. 8 means that the expression state shown on the left side is accelerated more than the expression state shown on the right side is. The inequality sign with an equal sign “≧” shows that the expression state shown on the left side is accelerated more than or equal to the expression state shown on the right side.

Hereinafter, the present invention is more specifically described with reference to Examples; however, the present invention is not limited thereto.

### EXAMPLES

### Preparation of Biological Samples

In the following examples, the biological samples obtained as follows were used. First, pathological diagnosis of thyroid lesion tissues extracted from patients with thyroid tumors by surgery was performed. These tissues were classified into one of the cases of adenomatous goiter, follicular adenoma, follicular carcinoma (including poorly-differentiated carcinoma), and papillary carcinoma. These tissues were used as biological samples and cryopreserved at −80° C.

### Example 1

### 1. Measurement of Gene Expression Level with Microarray

As the Learning set, tissue specimens of 25 cases which was pathologically classified into follicular neoplasm (11 cases with follicular adenoma and 14 cases with follicular carcinoma) were used. RNAs (100 to 200 ng) extracted from the specimens were amplified with GeneChip (registered trademark) 3′ IVT Express Kit (Affymetrix Inc.). The amplified RNAs were added to GeneChip (registered trademark) Human Genome U133 Plus 2.0 Array (Affymetrix Inc.) to react them at 65° C. for 16 hours. The RNAs were hybridized to probes on the array. Thereafter, the hybridized probes were stained using GeneChip (registered trademark) Hybridization, Wash, and Stain Kit (Affymetrix Inc.). Subsequently, the luminescence signals were detected using Genechip (registered trademark) Scanner 3000 (Affymetrix Inc.).

### 2. Marker Selection

The signal value data (CELL file) obtained by a microarray was normalized by MASS using Expression Console (Affymetrix Inc.). Thereafter, the probe whose function was not clear and which had no name was removed. As for probes having overlapping genes, probes other than the probe having the highest signal were removed. As for the signal values of the remaining probes, log-conversion in the base of 2 was performed to calculate CV values of the probes. The top 5000 genes with significant change among the 25 cases (11 cases with follicular adenoma and 14 cases with follicular carcinoma) were extracted. The 5000 genes were Z-scored. On the basis of the results, unsupervised cluster analysis was performed using cluster 3.0 (open source, obtained from the following web site; http://bonsai.hgc.jp/-mdehoonisoftware/cluster/). Specifically, the analysis procedure is as follows. The Cluster ring tab in the hierarchy of Cluster 3.0 was opened. The box of [Cluster] of [Genes] and the box of [Cluster] of [Arrays] were checked. As for Similarity Metric, the box of [Correlation(Centered)] in [Genes] and [Arrays] was selected. As for Clustering Method, the box of [Centroid linkage] was selected. Thus, the cluster analysis was conducted. The results of the cluster analysis were analyzed using Java Treeview (open source, obtained from the following web site; http://sourceforge.net/projects/jtreeview/). As a result of the cluster analysis using the 5000 genes, it is found that the 25 cases are classified into three groups (Clusters 1, 2, and 3).

Subsequently, in order to narrow down the genes, 37 genes were extracted as genes in which the expression of the genes is increased according to the tree diagram of the gene side. Then, the unsupervised cluster analysis was performed using only the 37 genes thus extracted, and the 25 cases could be classified into three groups. The results are shown in FIG. 1. As shown in the region surrounded by a square in FIG. 1, in Cluster 1 the expressions of 11 genes: AIF1L, CDH16, FAM162B, FGFR2, GJB6, KCNJ13, KIAA1467, SLC25A15, TFCP2L1, TFF3, and TMEM171 are increased, in Cluster 2 the expressions of 17 genes: C4orf10, CCDC8, CD22, FAM125A, FAM174B, FBF1, GLB1L2, LOC644613, MAP2K2, MTG1, PFKL, PTDSS2, SF3A2, SLC2A11, VILL, VSIG2, and WDR18 are increased, and in Cluster 3 the expression of 9 genes: ANXA1, C13orf33, CYP1B1, FAP, FN1, IL17RD, PDLIM4, RUNX2, and TIMP1 are increased. Hereinafter, the gene clusters in which the expression is increased in Clusters 1, 2, and 3 are referred to as “first gene cluster”, “second gene cluster”, and “third gene cluster”, respectively. Probe IDs of the genes are as shown in Table 1.

Among the 25 cases, 9 cases were classified into Cluster 1, 7 cases were classified into Cluster 2, and 9 cases were classified into Cluster 3. The gene expression-based classification was correlated with the pathology classification. As a result, it was found that follicular adenomas, i.e., benign tumors were concentrated in Cluster 1 (8 cases of 9 cases), and follicular carcinomas, i.e., malignant tumors were concentrated in Clusters 2 and 3 (Cluster 2: 6 cases of 7 cases, Cluster 3: 7 cases of 9 cases). The results are shown in Table 2 (conveniently, the tumor of the case classified into Cluster 2 is referred to as “first malignant tumor”, and the tumor of the case classified into Cluster 3 is referred to as “second malignant tumor”).

TABLE 2

| | | 37 genes | | |
|---|---|---|---|---|
| | | Cluster 1 | Cluster 2 | Cluster 3 |
| | | Benign | First malignant | Second malignant |
| Cluster analysis | | tumor | tumor | tumor |
| Pathological | Follicular | 8 | 1 | 2 |
| classification | adenoma | | | |
| | Follicular | 1 | 6 | 7 |
| | carcinoma | | | |

Accordingly, a total of 37 genes obtained by combining genes in which the expression was increased in Clusters 1, 2 and 3 were used and follicular neoplasms were classified into three groups. The cases classified into Cluster 1 were determined as follicular adenoma, and the cases classified into Cluster 2 or 3 were determined as follicular carcinoma.

### Example 2

Twenty genes below were extracted from the 37 genes and the cluster analysis was performed in the same manner as in Example 1. As a result, 25 cases were classified into three groups in the same manner as in Example 1. In Cluster 1 the expressions of 6 genes: AIF1L, FAM162B, FGFR2, GJB6, KCNJ13, and TFF3 are increased, in Cluster 2 the expressions of 7 genes: FAM125A, MAP2K2, MTG1, PFKL, PTDSS2, SF3A2, and WDR18 are increased, and in Cluster 3 the expressions of 7 genes: ANXA1, CYP1B1, FAP, IL17RD, PDLIM4, RUNX2, and TIMP1 are increased. Among the 25 cases, 11 cases were classified into Cluster 1, 6 cases were classified into Cluster 2, and 8 cases were classified into Cluster 3. The results obtained by correlating the gene expression-based classification with the pathological classification are shown in Table 3.

TABLE 3

| | | 20 genes | | |
|---|---|---|---|---|
| | | Cluster 1 | Cluster 2 | Cluster 3 |
| | | Benign | First malignant | Second malignant |
| Cluster analysis | | tumor | tumor | tumor |
| Pathological | Follicular | 8 | 1 | 2 |
| classification | adenoma | | | |
| | Follicular | 3 | 5 | 6 |
| | carcinoma | | | |

As shown in Table 3, it was found that follicular adenomas were concentrated in Cluster 1 (8 cases of 11 cases) and follicular carcinomas were concentrated in Clusters 2 and 3 (Cluster 2: 5 cases of 6 cases, Cluster 3: 6 cases of 8 cases). Accordingly, a total of 20 genes obtained by combining genes in which the expression was increased in Clusters 1, 2 and 3 were used and follicular neoplasms were classified into three groups. The cases classified into Cluster 1 were determined as follicular adenoma, and the cases classified into Cluster 2 or 3 were determined as follicular carcinoma.

### Example 3

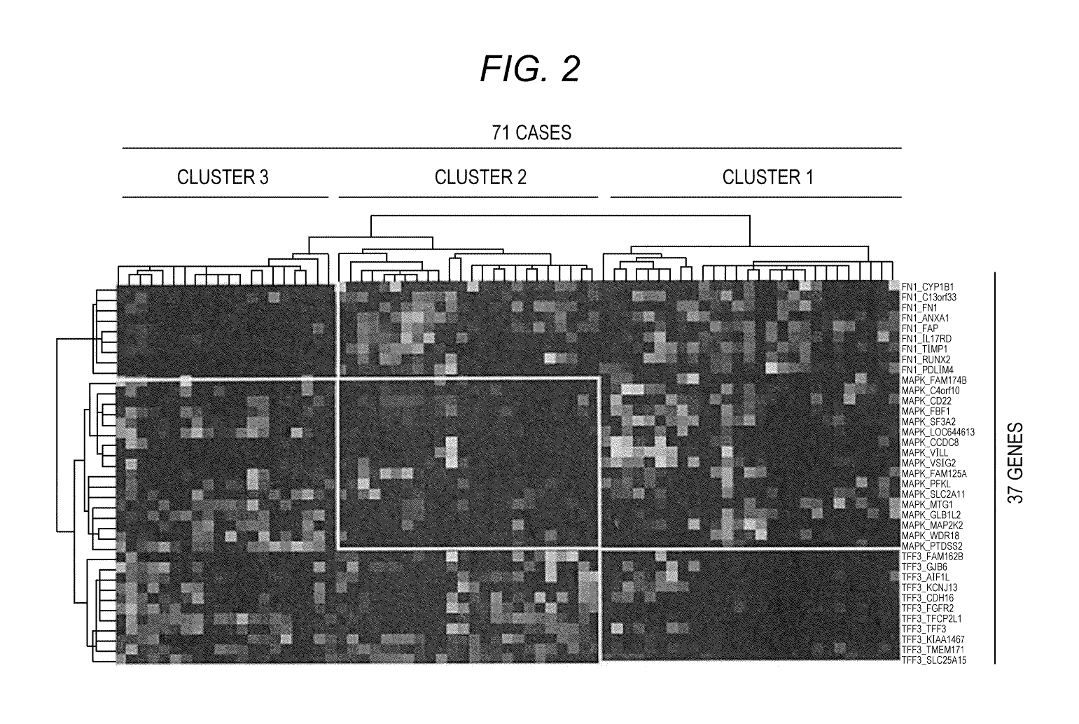

It was examined whether the cluster was reproduced even when a specimen different from the tissue specimen used in Example 1 was used. As the Validation set, tissue specimens in 71 cases different from the Learning set (33 cases with follicular adenoma, 38 cases with follicular carcinoma) were used. RNAs were extracted from the specimens and the expression levels of the 37 genes were measured using the microarray in the same manner as described in Example 1. The signal value data (CELL file) obtained by a microarray was normalized by MASS using Expression Console (Affymetrix Inc.). The signal values of the 37 genes selected in Example 1 were extracted. The resulting values were subjected to log-conversion in the base of 2 and further the log-converted values were Z-scored. On the basis of the results, unsupervised cluster analysis was performed using cluster 3.0. The procedure of cluster analysis is the same as in Example 1. As a result, the 71 cases could be classified into three groups (refer to FIG. 2). Among the 71 cases, 27 cases were classified into Cluster 1, 24 cases were classified into Cluster 2, and 20 cases were classified into Cluster 3. The results obtained by correlating the gene expression-based classification with the pathological classification are shown in Table 4.

TABLE 4

| | | 37 genes | | |
|---|---|---|---|---|
| | | Cluster 1 | Cluster 2 | Cluster 3 |
| | | Benign | First malignant | Second malignant |
| Cluster analysis | | tumor | tumor | tumor |
| Pathological | Follicular | 21 | 6 | 6 |
| classification | adenoma | | | |
| | Follicular | 6 | 18 | 14 |
| | carcinoma | | | |

As shown in Table 4, it was found that follicular adenomas were concentrated in Cluster 1 (21 cases of 27 cases) and follicular carcinomas were concentrated in Clusters 2 and 3 (Cluster 2: 18 cases of 24 cases, Cluster 3: 14 cases of 20 cases). Thus, as for the determination of behavior of thyroid tumor based on the analysis of the expression levels of the 37 genes, good reproducibility was obtained. Even when the 20 genes were used, the same tendency was observed.

### Example 4

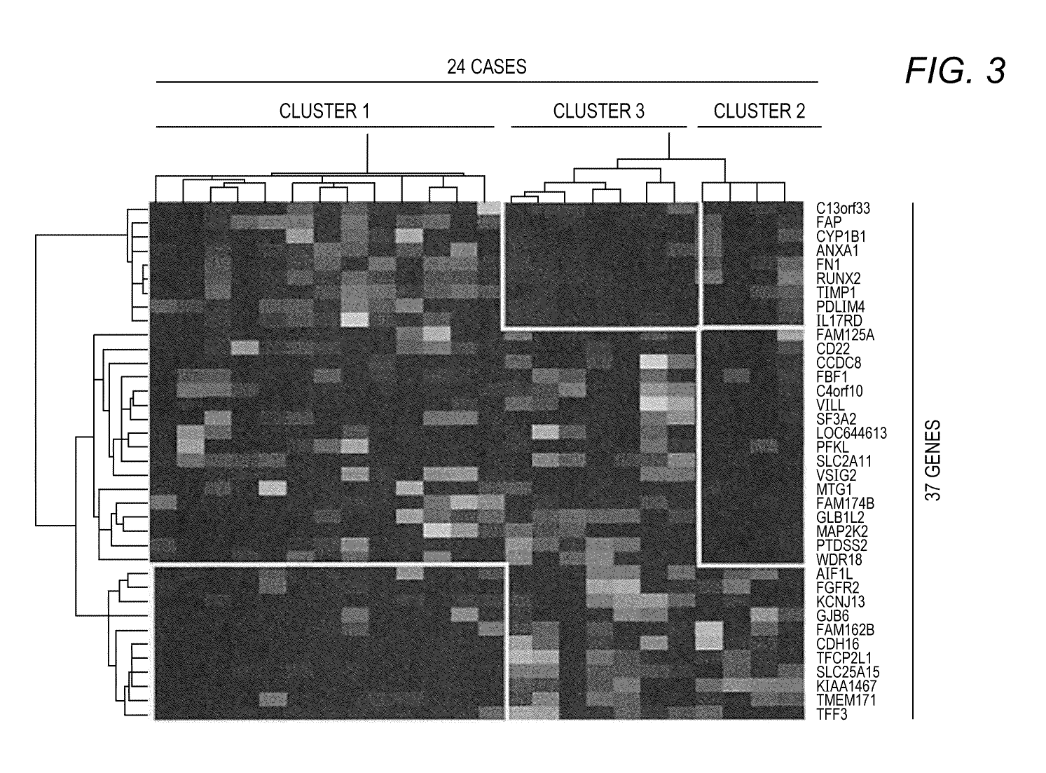

It was examined whether the cluster was reproduced even when adenomatous goiter and papillary carcinoma as specimens were added to follicular adenoma and follicular carcinoma. As for 14 cases with benign tumor (8 cases with adenomatous goiter and 6 cases with follicular adenoma) and 10 cases with malignant tumors (4 cases with papillary carcinoma and 6 cases with follicular carcinoma), RNAs were extracted from the tissue specimens and the expression levels of the 37 genes were measured using the microarray in the same manner as described in Example 1. The procedure of cluster analysis is the same as in Example 1. As a result, the 24 cases could be classified into three groups (refer to FIG. 3). Among the 24 cases, 13 cases were classified into Cluster 1, 4 cases were classified into Cluster 2, and 7 cases were classified into Cluster 3. The results obtained by correlating the gene expression-based classification with the pathological classification are shown in Table 5.

TABLE 5

| | | 37 genes | | |
|---|---|---|---|---|
| | | Cluster 1 | Cluster 2 | Cluster 3 |
| | | Benign | First malignant | Second malignant |
| Cluster analysis | | tumor | tumor | tumor |
| Pathological | Benign | 13 | 0 | 1 |
| classification | tumor | | | |
| | Malignant | 0 | 4 | 6 |
| | tumor | | | |

As shown in Table 5, it was found that benign tumors were concentrated in Cluster 1 (13 cases of 13 cases) and malignant tumors were concentrated in Clusters 2 and 3 (Cluster 2: 4 cases of 4 cases, Cluster 3: 6 cases of 7 cases). Thus, even if tumor tissues of different types from those of the tumor tissues used when extracting the 37 genes were used as specimens, good reproducibility was obtained in the determination of behavior of thyroid tumor. Accordingly, the 37 genes were used and thyroid tumors were classified into three groups. It was found that the cases classified into Cluster 1 were determined as benign tumor, and the cases classified into Cluster 2 or 3 were determined as malignant tumor.

### Example 5

In this example, in order to perform cluster classification of each case (determination of behavior of tumor) more accurately, it is intended to construct a determination formula for the expression levels of the 37 genes. As biological samples, tissue specimens in 45 cases with benign tumor (adenomatous goiter and follicular adenoma) and 42 cases with malignant tumors (papillary carcinoma and follicular carcinoma) were used. RNAs were extracted from the specimens and the expression levels of the 37 genes (signal values from probes) were measured using the microarray in the same manner as described in Example 1. The signal values were subjected to log-conversion in the base of 2. In this example, averages of the log-converted signal values as for the first, second, and third gene clusters were compared, and the determination formula for classifying each case into each Cluster was tried (hereinafter, also referred to as “determination formula (I)”).

Here, the procedure of determination by the determination formula (I) will be described. First, an average of the signal values in each cluster of the first, second, and third gene clusters was calculated. Then, the resulting averages of the first, second, and third gene clusters were compared in the respective cases. The respective cases were classified into the cluster to which the gene cluster having the highest average (that is, the gene cluster in which the expression level is the most increased) belongs.

The determination formula (I) was applied to all the 87 cases and the cluster classification was performed. As a result, among the 87 cases, 38 cases were classified into Cluster 1, 23 cases were classified into Cluster 2, and 26 cases were classified into Cluster 3. The results obtained by correlating the determination formula-based classification with the pathological classification are shown in Table 6.

TABLE 6

| | | 37 genes | | |
|---|---|---|---|---|
| | | Cluster 1 | Cluster 2 | Cluster 3 |
| Determination formula | | Benign | First malignant | Second malignant |
| (I) | | tumor | tumor | tumor |
| Pathological | Benign | 32 | 6 | 7 |
| classification | tumor | | | |
| | Malignant | 6 | 17 | 19 |
| | tumor | | | |

As shown in Table 6, it was found that benign tumors were concentrated in Cluster 1 (32 cases of 38 cases) and malignant tumors were concentrated in Clusters 2 and 3 (Cluster 2: 17 cases of 23 cases, Cluster 3: 19 cases of 26 cases). As the performance of determination by the determination formula (I), a good performance such that the sensitivity was 86%, the specificity was 71%, the concordance rate was 78%, the PPV was 73%, and the NPV was 84% was obtained. The determination method using the 37 genes showed excellent specificity and PPV as compared with the determination method by the Afirma Thyroid FNA Analysis (a product commercially available from Veracyte, Inc.).

### Example 6

The 20 genes used in Example 2 were used and classifications of 45 cases with benign tumors (adenomatous goiter and follicular adenoma) and 42 cases with malignant tumors (papillary carcinoma and follicular carcinoma) were performed by the determination formula (I) in the same manner as described in Example 5. As a result, among the 87 cases, 43 cases were classified into Cluster 1, 25 cases were classified into Cluster 2, and 19 cases were classified into Cluster 3. The results obtained by correlating the determination formula-based classification with the pathological classification are shown in Table 7.

TABLE 7

| | | 20 genes | | |
|---|---|---|---|---|
| | | Cluster 1 | Cluster 2 | Cluster 3 |
| Determination formula | | Benign | First malignant | Second malignant |
| (I) | | tumor | tumor | tumor |
| Pathological | Benign | 34 | 7 | 4 |
| classification | tumor | | | |
| | Malignant | 9 | 18 | 15 |
| | tumor | | | |

As shown in Table 7, it was found that benign tumors were concentrated in Cluster 1 (34 cases of 43 cases) and malignant tumors were concentrated in Clusters 2 and 3 (Cluster 2: 18 cases of 25 cases, Cluster 3: 15 cases of 19 cases). As the performance of determination by the determination formula (I), a good performance such that the sensitivity was 79%, the specificity was 76%, the concordance rate was 77%, the PPV was 75%, and the NPV was 79% was obtained. The determination method using the 20 genes showed excellent specificity and PPV as compared with the determination method by the Afirma Thyroid FNA Analysis (a product commercially available from Veracyte, Inc.).

### Example 7

In this example, it is intended to construct a determination formula different from that of Example 5. As biological samples, tissue specimens in 45 cases with benign tumor (adenomatous goiter and follicular adenoma) and 42 cases with malignant tumors (papillary carcinoma and follicular carcinoma) were used. RNAs were extracted from the specimens and the expression levels of the 37 genes (signal values from probes) were measured using the microarray in the same manner as described in Example 1. In this example, the determination formula to classify the genes based on the gene expression patterns of the cases calculated by the primary linear equation and distances from the three clusters classified in Example 1 was tried (hereinafter, the formula is referred to as “determination formula (II)”).

Here, the procedure of determination by the determination formula (II) will be described. In the determination formula (II), the signal values of the 37 genes as for the Learning set used in Example 1 were plotted. Differences among the expression levels for the respective clusters were used to classify the genes into three clusters. The three clusters are the same as Clusters 1, 2, and 3 in Example 1. Then, an average of signal values of the clusters was calculated. A Euclid distance between the average of the clusters and data of signal values of the 87 cases was calculated by the primary linear equation. The distances from the clusters in the respective cases were compared. The respective cases were classified into the cluster with the closest distance. As a result, among the 87 cases, 33 cases were classified into Cluster 1, 35 cases were classified into Cluster 2, and 19 cases were classified into Cluster 3. The results obtained by correlating the determination formula-based classification with the pathological classification are shown in Table 8.

TABLE 8

| | | 37 genes | | |
|---|---|---|---|---|
| | | Cluster 1 | Cluster 2 | Cluster 3 |
| Determination formula | | Benign | First malignant | Second malignant |
| (II) | | tumor | tumor | tumor |
| Pathological | Benign | 29 | 10 | 6 |
| classification | tumor | | | |
| | Malignant | 4 | 25 | 13 |
| | tumor | | | |

As shown in Table 8, it was found that benign tumors were concentrated in Cluster 1 (29 cases of 33 cases) and malignant tumors were concentrated in Clusters 2 and 3 (Cluster 2: 25 cases of 35 cases, Cluster 3: 13 cases of 19 cases). As the performance of determination by the determination formula (II), a good performance such that the sensitivity was 90%, the specificity was 64%, the concordance rate was 77%, the PPV was 70%, and the NPV was 88% was obtained. The determination method using the 37 genes showed excellent specificity and PPV as compared with the determination method by the Afirma Thyroid FNA Analysis (a product commercially available from Veracyte, Inc.).

### Example 8

The 20 genes used in Example 2 were used and classifications of 45 cases with benign tumors (adenomatous goiter and follicular adenoma) and 42 cases with malignant tumors (papillary carcinoma and follicular carcinoma) were performed by the determination formula (II) in the same manner as described in Example 7. As a result, among the 87 cases, 33 cases were classified into Cluster 1, 29 cases were classified into Cluster 2, and 25 cases were classified into Cluster 3. The results obtained by correlating the determination formula-based classification with the pathological classification are shown in Table 9.

TABLE 9

| | | 20 genes | | |
|---|---|---|---|---|
| | | Cluster 1 | Cluster 2 | Cluster 3 |
| Determination formula | | Benign | First malignant | Second malignant |
| (II) | | tumor | tumor | tumor |
| Pathological | Benign | 27 | 11 | 7 |
| classification | tumor | | | |
| | Malignant | 6 | 18 | 18 |
| | tumor | | | |

As shown in Table 9, it was found that benign tumors were concentrated in Cluster 1 (27 cases of 33 cases) and malignant tumors were concentrated in Clusters 2 and 3 (Cluster 2: 18 cases of 29 cases, Cluster 3: 18 cases of 25 cases). As the performance of determination by the determination formula (II), a good performance such that the sensitivity was 86%, the specificity was 60%, the concordance rate was 72%, the PPV was 67%, and the NPV was 82% was obtained. The determination method using the 20 genes showed excellent specificity and PPV as compared with the determination method by the Afirma Thyroid FNA Analysis (a product commercially available from Veracyte, Inc.).

### Example 9

In this example, a difference between Cluster 2 and Cluster 3 was examined from the viewpoint of the prognosis in malignant tumor. Among follicular carcinoma, poorly-differentiated carcinoma, widely invasive-type follicular carcinoma, and follicular carcinoma with vascular invasion are generally known as poor-prognostic malignant tumors. Here, the cases used in Example 7 include 15 cases which are malignant tumors with poor prognosis of the cases. Then, it was examined into which Cluster these cases were classified in Example 7. The results are shown in Table 10.

TABLE 10

| | | 37 genes | | |
|---|---|---|---|---|
| | | Cluster 1 | Cluster 2 | Cluster 3 |
| Determination formula | | Benign | First malignant | Second malignant |
| (II) | | tumor | tumor | tumor |
| Pathological | Benign | 0 | 0 | 0 |
| classification | tumor | | | |
| | Malignant | 0 | 11 | 4 |
| | tumor | | | |

As shown in Table 10, the number of cases classified into Cluster 1 was 0, the number of cases classified into Cluster 2 was 11, and the number of cases classified into Cluster 3 was 4. Thus, a greater number of cases with poor prognosis were concentrated in Cluster 2. This suggested that the case classified into Cluster 2 (the first malignant tumor) had a higher possibility of being a malignant tumor with poor prognosis as compared with the case classified into Cluster 3 (the second malignant tumor).

What is claimed is:

1. A system adapted to a method for determining behavior of thyroid tumor, comprising:
a processor, and
a memory, under control of said processor, including software instructions adapted to enable the system to perform operations comprising:
determining whether a thyroid tumor in a subject is benign or malignant based on the results of first, second, and third measurements;
wherein the expression levels of at least 6 genes selected from the group consisting of AIF1L, CDH16, FAM162B, FGFR2, GJB6, KCNJ13, KIAA1467, SLC25A15, TFCP2L1, TFF3, and TMEM171 in a sample collected from the subject are measured in the first measurement,
the expression levels of at least 7 genes selected from the group consisting of C4orf10, CCDC8, CD22, FAM125A, FAM174B, FBF1, GLB1L2, LOC644613, MAP2K2, MTG1, PFKL, PTDSS2, SF3A2, SLC2A11, VILL, VSIG2, and WDR18 in the sample are measured in the second measurement, and
the expression levels of at least 7 genes selected from the group consisting of ANXA1, C13orf33, CYP1B1, FAP, FN1, IL17RD, PDLIM4, RUNX2, and TIMP1 in the sample are measured in the third measurement.

2. The system according to claim 1, wherein
in the operations,
when the expression state of the genes measured in the first measurement is more than or equal to the expression state of the genes measured in the second measurement, and when the expression state of the genes measured in the first measurement is more than or equal to the expression state of the genes measured in the third measurements, the thyroid tumor of the subject is determined to be benign, and
when the expression state of the genes measured in either the second or third measurement is more than the expression state of the genes measured in the first measurement, the thyroid tumor of the subject is determined to be malignant.

3. The system according to claim 1, wherein
in the operations,
when the expression state of the genes measured in the first measurement is more than or equal to the expression state of the genes measured in the second measurement, and when the expression state of the genes measured in the first measurement is more than or equal to the expression state of the genes measured in the third measurement, the thyroid tumor of the subject is determined to be benign,
when the expression state of the genes measured in the second measurement is more than the genes measured in the first measurement, and when the expression state of the genes measured in the second measurement is more than or equal to the expression state of the genes measured in the third measurement, the thyroid tumor of the subject is determined to a first malignant tumor, and
when the expression state of the genes measured in the third measurement is more than the expression states of the genes measured in the first and second measurements, the thyroid tumor of the subject is determined to a second malignant tumor.

4. The system according to claim 1, wherein
the benign tumor is at least one of follicular adenoma, adenomatous nodules, and cysts.

5. The system according to claim 1, wherein
the malignant tumor is at least one of follicular carcinoma, papillary carcinoma, poorly-differentiated carcinoma, and undifferentiated carcinoma.

6. The system according to claim 3, wherein
the first malignant tumor is a malignant tumor with poor prognosis.

7. The system according to claim 3, wherein
the second malignant tumor is a malignant tumor with good prognosis.

8. The system according to claim 3, wherein
the first malignant tumor is at least one of undifferentiated carcinoma, poorly-differentiated carcinoma, and follicular carcinoma with widely invasive and/or vascular invasion.

9. The system according to claim 3, wherein
the second malignant tumor is at least one of papillary carcinoma and follicular carcinoma without widely invasive and vascular invasion.

10. The system according to claim 1, wherein
the expression levels of at least 6 genes: AIF1L, FAM162B, FGFR2, GJB6, KIAA1467, and TFF3 are measured in the first measurement.

11. The system according to claim 1, wherein
the expression levels of at least 7 genes: FAM174B, MAP2K2, MTG1, PFKL, PTDSS2, SF3A2, and WDR18 are measured in the second measurement.

12. The system according to claim 1, wherein
the expression levels of at least 7 genes: ANXA1, CYP1B1, FAP, IL17RD, PDLIM4, RUNX2, and TIMP1 are measured in the third measurement.

13. The system according to claim 1, wherein
a microarray or a nucleic acid amplification method is used to measure the gene expression levels in the first, second, and third measurements.

14. The system according to claim 1, wherein
the sample contains cells derived from the thyroid of the subject.

15. The system according to claim 1, further comprising a microarray and a microarray scanner to perform the first, second and third measurements,
the microarray scanner being connected to the processor to transmit a signal detected from the microarray.

16. The system according to claim 2, wherein
the expression state of the genes measured in the first measurement is a measure of central tendency of the expression levels of genes measured in the first measurement,
the expression state of the genes measured in the second measurement is a measure of central tendency of the expression levels of genes measured in the second measurement, and
the expression state of the genes measured in the third measurement is a measure of central tendency of the expression levels of genes measured in the third measurement.

17. A computer program product for enabling a computer to determine behavior of thyroid tumor, comprising:
a computer readable medium, and
software instructions, on the computer readable medium, for enabling the computer to perform predetermined operations comprising:
determining whether a thyroid tumor in a subject is benign or malignant based on the results of first, second, and third measurements, wherein
the expression levels of at least 6 genes selected from the group consisting of AIF1L, CDH16, FAM162B, FGFR2, GJB6, KCNJ13, KIAA1467, SLC25A15, TFCP2L1, TFF3, and TMEM171 in a sample collected from the subject are measured in the first measurement,
the expression levels of at least 7 genes selected from the group consisting of C4orf10, CCDC8, CD22, FAM125A, FAM174B, FBF1, GLB1L2, LOC644613, MAP2K2, MTG1, PFKL, PTDSS2, SF3A2, SLC2A11, VILL, VSIG2, and WDR18 in the sample are measured in the second measurement, and the expression levels of at least 7 genes selected from the group consisting of ANXA1, C13orf33, CYP1B1, FAP, FN1, IL17RD, PDLIM4, RUNX2, and TIMP1 in the sample are measured in the third measurement.

18. The computer program product according to claim 17, wherein
in the operations,
when the expression state of the genes measured in the first measurement is more than or equal to the expression state of the genes measured in the second measurement, and when the expression state of the genes measured in the first measurement is more than or equal to the expression state of the genes measured in the third measurements, the thyroid tumor of the subject is determined to be benign, and
when the expression state of the genes measured in either the second or third measurement is more than the expression state of the genes measured in the first measurement, the thyroid tumor of the subject is determined to be malignant.

19. The computer program product according to claim 17, wherein
in the operations,
when the expression state of the genes measured in the first measurement is more than or equal to the expression state of the genes measured in the second measurement, and when the expression state of the genes measured in the first measurement is more than or equal to the expression state of the genes measured in the third measurement, the thyroid tumor of the subject is determined to be benign,
when the expression state of the genes measured in the second measurement is more than the genes measured in the first measurement, and when the expression state of the genes measured in the second measurement is more than or equal to the expression state of the genes measured in the third measurement, the thyroid tumor of the subject is determined to a first malignant tumor, and
when the expression state of the genes measured in the third measurement is more than the expression states of the genes measured in the first and second measurements, the thyroid tumor of the subject is determined to a second malignant tumor.

20. A method for determining behavior of thyroid tumor, comprising:
applying a sample collected from a subject with a thyroid tumor to a microarray capable of measuring the expression levels of the following genes (a) to (c):
(a) at least 6 genes selected from the group consisting of AIF1L, CDH16, FAM162B, FGFR2, GJB6, KCNJ13, KIAA1467, SLC25A15, TFCP2L1, TFF3, and TMEM171;
(b) at least 7 genes selected from the group consisting of C4orf10, CCDC8, CD22, FAM125A, FAM174B, FBF1, GLB1L2, LOC644613, MAP2K2, MTG1, PFKL, PTDSS2, SF3A2, SLC2A11, VILL, VSIG2, and WDR18; and
(c) at least 7 genes selected from the group consisting of ANXA1, C13orf33, CYP1B1, FAP, FN1, IL17RD, PDLIM4, RUNX2, and TIMP1;
detecting optical information about the expression levels of the genes (a) to (c) obtained from the microarray using a microarray scanner after the applying step;
sending the optical information detected in the detecting step to a CPU of a computer connected to the microarray scanner;
calculating the expression levels of the genes (a) to (c), based on the optical information by the CPU; and
determining whether the thyroid tumor in the subject is benign or malignant using the values calculated by the CPU in the calculating step.
